(19) United States
(12) Patent Application Publication
(10) Pub. No.: US 2023/0089140 A1
(43) Pub. Date: Mar. 23, 2023

(54) MACHINE LEARNING TECHNIQUES USING SEGMENT-WISE REPRESENTATIONS OF INPUT FEATURE REPRESENTATION SEGMENTS

(71) Applicant: Optum Services (Ireland) Limited, Dublin (IE)

(72) Inventors: Ahmed Selim, Dublin (IE); Mostafa Bayomi, Dublin (IE); Kieran O'Donoghue, Dublin (IE); Michael Bridges, Dublin (IE)

(21) Appl. No.: 17/648,382

(22) Filed: Jan. 19, 2022

Related U.S. Application Data
(60) Provisional application No. 63/246,103, filed on Sep. 20, 2021.

(51) Int. Cl. G16B 20/20; G16B 40/00

(52) U.S. Cl. CPC G16B 20/20; G16B 40/00

(57) ABSTRACT

Various embodiments of the present invention provide methods, apparatus, systems, computing devices, computing entities, and/or the like for performing health-related predictive data analysis. Certain embodiments of the present invention utilize systems, methods, and computer program products that perform predictive data analysis by using at least one of shared segment embedding machine learning models or transformer-based machine learning models.

### CROSS-REFERENCES TO RELATED APPLICATION(S)

The present application claims priority to the U.S. Provisional Patent Application No. 63/246,103, filed on Sep. 20, 2021, which is incorporated by reference herein in its entirety.

### BACKGROUND

Various embodiments of the present invention address technical challenges related to performing health-related predictive data analysis. Various embodiments of the present invention address the shortcomings of existing predictive data analysis systems and disclose various techniques for efficiently and reliably performing predictive data analysis.

### BRIEF SUMMARY

In general, embodiments of the present invention provide methods, apparatus, systems, computing devices, computing entities, and/or the like for performing health-related predictive data analysis. Certain embodiments of the present invention utilize systems, methods, and computer program products that perform predictive data analysis by using at least one of shared segment embedding machine learning models or transformer-based machine learning models.

In accordance with one aspect, a method is provided. In one embodiment, the method comprises: determining, based at least in part on the initial input feature representation, an ordered sequence of n input feature representation values, wherein: (i) the initial input feature representation is a fixed-size representation of an input feature comprising g feature values, (ii) each feature value corresponds to a genetic variant identifier of g genetic variant identifiers, (iii) each genetic variant identifier is associated with a chromosome designation of c chromosome designations and a corresponding variant-related subsequence of the ordered sequence, and (iv) each chromosome designation is associated with a chromosome-related subsequence of the ordered sequence; generating, based at least in part on the ordered sequence, c input feature representation super-segments, wherein each input feature representation segment is associated with a corresponding chromosome designation and comprises the chromosome-related subsequence for the corresponding chromosome designation; generating, based at least in part on the c input feature representation super-segments, m input feature representation segments of the ordered sequence, wherein the m input feature representation segments comprise, for each chromosome designation, a chromosome-related segment subset of the m input feature representation segments that comprises those input feature representation segments that are generated by segmentizing the input feature representation super-segment for the chromosome designation; for each input feature representation segment, determining, using a shared segment embedding machine learning model and based at least in part on the input feature representation segment, a segment-wise representation of the input feature representation segment; determine, using a transformer-based machine learning model and based at least in part on each segment-wise representation, a multi-segment input feature representation of the input feature; generating, using the one or more processors and based at least in part on the multi-segment input feature representation and using a downstream prediction machine learning model, a multi-segment prediction; and performing, using the one or more processors, one or more prediction-based actions based at least in part on the multi-segment prediction.

In accordance with another aspect, a computer program product is provided. The computer program product may comprise at least one computer-readable storage medium having computer-readable program code portions stored therein, the computer-readable program code portions comprising executable portions configured to: determine, based at least in part on the initial input feature representation, an ordered sequence of n input feature representation values, wherein: (i) the initial input feature representation is a fixed-size representation of an input feature comprising g feature values, (ii) each feature value corresponds to a genetic variant identifier of g genetic variant identifiers, (iii) each genetic variant identifier is associated with a chromosome designation of c chromosome designations and a corresponding variant-related subsequence of the ordered sequence, and (iv) each chromosome designation is associated with a chromosome-related subsequence of the ordered sequence; generate, based at least in part on the ordered sequence, c input feature representation super-segments, wherein each input feature representation segment is associated with a corresponding chromosome designation and comprises the chromosome-related subsequence for the corresponding chromosome designation; generate, based at least in part on the c input feature representation super-segments, m input feature representation segments of the ordered sequence, wherein the m input feature representation segments comprise, for each chromosome designation, a chromosome-related segment subset of the m input feature representation segments that comprises those input feature representation segments that are generated by segmentizing the input feature representation super-segment for the chromosome designation; for each input feature representation segment, determine, using a shared segment embedding machine learning model and based at least in part on the input feature representation segment, a segment-wise representation of the input feature representation segment; determine, using a transformer-based machine learning model and based at least in part on each segment-wise representation, a multi-segment input feature representation of the input feature; generate, using the one or more processors and based at least in part on the multi-segment input feature representation and using a downstream prediction machine learning model, a multi-segment prediction; and perform, using the one or more processors, one or more prediction-based actions based at least in part on the multi-segment prediction.

In accordance with yet another aspect, an apparatus comprising at least one processor and at least one memory including computer program code is provided. In one embodiment, the at least one memory and the computer program code may be configured to, with the processor, cause the apparatus to: determine, based at least in part on the initial input feature representation, an ordered sequence of n input feature representation values, wherein: (i) the initial input feature representation is a fixed-size representation of an input feature comprising g feature values, (ii) each feature value corresponds to a genetic variant identifier of g genetic variant identifiers, (iii) each genetic variant identifier is associated with a chromosome designation of c chromosome designations and a corresponding variant-related subsequence of the ordered sequence, and (iv) each chromosome designation is associated with a chromosome-related subsequence of the ordered sequence; generate, based at least in part on the ordered sequence, c input feature representation super-segments, wherein each input feature representation segment is associated with a corresponding chromosome designation and comprises the chromosome-related subsequence for the corresponding chromosome designation; generate, based at least in part on the c input feature representation super-segments, m input feature representation segments of the ordered sequence, wherein the m input feature representation segments comprise, for each chromosome designation, a chromosome-related segment subset of the m input feature representation segments that comprises those input feature representation segments that are generated by segmentizing the input feature representation super-segment for the chromosome designation; for each input feature representation segment, determine, using a shared segment embedding machine learning model and based at least in part on the input feature representation segment, a segment-wise representation of the input feature representation segment; determine, using a transformer-based machine learning model and based at least in part on each segment-wise representation, a multi-segment input feature representation of the input feature; generate, using the one or more processors and based at least in part on the multi-segment input feature representation and using a downstream prediction machine learning model, a multi-segment prediction; and perform, using the one or more processors, one or more prediction-based actions based at least in part on the multi-segment prediction.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example external computing entity in accordance with some embodiments discussed herein.

FIG. 4 is a flowchart diagram of an example process for generating a multi-segment prediction for an input feature in accordance with some embodiments discussed herein.

FIG. 5 is a flowchart diagram of an example process for generating an initial input feature representation for an input feature in accordance with some embodiments discussed herein.

FIG. 6 provides an operational example of an example of image regions for an image representation in accordance with some embodiments discussed herein.

FIGS. 7-8 provide operational examples of an example image representations for a plurality of input feature type designations in accordance with some embodiments discussed herein.

FIG. 9 provides an operational example of a tensor representation in accordance with some embodiments discussed herein.

FIG. 10 provides an operational example of a plurality of positional encoding maps in accordance with some embodiments discussed herein.

FIG. 11 provides an operational example of a tensor representation with the plurality of positional encoding maps in accordance with some embodiments discussed herein.

FIG. 12 is a flowchart diagram of an example process for generating a differential image representation in accordance with some embodiments discussed herein.

FIG. 13 provides an operational example of an example input feature for a first allele and second allele in accordance with some embodiments discussed herein.

FIGS. 14A-D provide operational examples of example image representations for an input feature type designation in accordance with some embodiments discussed herein.

FIG. 15 is a flowchart diagram of an example process for generating an intensity image representation in accordance with some embodiments discussed herein.

FIG. 16 is a flowchart diagram of an example process for generating a zygosity image representation in accordance with some embodiments discussed herein.

FIG. 17 provides an operational example of an example input feature for a dominant allele and minor allele in accordance with some embodiments discussed herein.

FIGS. 18-19 provide operational examples of an allele image representation in accordance with some embodiments discussed herein.

FIG. 20 provides an operational example of a zygosity image representation in accordance with some embodiments discussed herein.

FIG. 21 provides an operational example of a plurality of positional encoding maps in accordance with some embodiments discussed herein.

FIG. 22 provides an operational example of a tensor representation in accordance with some embodiments discussed herein.

FIG. 23 provides an operational example of an example input feature in accordance with some embodiments discussed herein.

FIG. 24 is a data flow diagram of an example process for generating a multi-segment input feature representation in accordance with some embodiments discussed herein.

FIG. 25 is a flowchart diagram of an example process for generating a set of input feature representation segments based at least in part on an initial input feature representation in accordance with some embodiments discussed herein.

FIG. 26 provides an operational example of a predictive output user interface in accordance with some embodiments discussed herein.

FIG. 27 provides an operational example of generating a multi-segment input feature representation in accordance with some embodiments discussed herein.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. Overview and Technical Advantages

Various embodiments of the present invention address technical challenges related to efficiently performing machine learning tasks on large datasets and/or on data-intensive datasets. As described below, in various embodiments of the present invention, a large and/or data-intensive dataset is converted into input feature representation super-segments and input feature representation segments, where the input feature representation super-segments are mapped to sentences and input feature representation segments are mapped to words. Then, segment-wise representations for input feature representation segments are provided to a transformer-based language model in accordance with the sentence-word hierarchy described above to generate multi-segment input feature representations that can then be used to perform efficient and effective predictive data analysis operations. This highlights a major technical advantage of the noted embodiments of the present invention: instead of processing an initial input feature representation as a whole, the noted embodiments of the present invention first generate m input feature representation segments of the initial input feature representation, and then process the m input feature representation segments using efficient and effective transformer-based language models. As a result, instead of performing the often excessively large computational task of processing the initial input feature representation as a whole and using an excessively large amount of computational resources and a large amount of processing time, various embodiments of the present invention divide the noted computational task into smaller computational sub-tasks that can be more manageably executed using transformer-based language models and by utilizing the sentence-word hierarchy described above. In this way, various embodiments of the present invention enable faster and less-resource-intensive processing of large machine learning tasks and/or data-intensive machine learning tasks by hierarchically segmenting input spaces and using the noted hierarchical segmentations to enable transformer-based encoding of the noted input spaces.

An exemplary application of various embodiments of the present invention relates to performing machine learning tasks on large-scale genomics data. Since the completion of the human genome program in 2003, an increasing amount of genomics data of different types are available. Large-scale sequencing programs, such as the UK's National Genomics Information Service and the “100,000 Genomes” project, exemplify the exponential increase in such data, which some authors have suggested will become the most prevalent field of big data. However, there is an even more fundamental concern, regarding how to represent genetic variants in a consistent format for ingestion by Deep Learning (DL) algorithms. For example, the most prevalent type of genetic data is arguably single-nucleotide polymorphisms (SNPs), arising from genome-wide association studies (GWAS) to investigate point mutations which may have casual associations with a specific disease, usually realized via case-control studies. Typically, the raw data from a whole-genome sequence (WGS) comprises approximately 3×10\hat{ }9 nucleotides and their corresponding quality scores. For a 30× coverage sequence, the FASTQ file would be roughly 100 GB in size, if uncompressed. In a typical variant calling, the resulting Binary Alignment Map (BAM) and Variant Call Format (VCF) files also feature high-dimensionality and can also be significant in size. As a concrete example, the DNA microarray component of the UK BioBank dataset illustrates this complexity: 850,000 variants were directly measured, with more than 90 million variants imputed using the Haplotype Reference Consortium. It is very challenging to an have an ML framework to ingest this massive amount of data, and to extract patterns related to downstream tasks. Due to the massive size of genomics data and software/hardware limitations, it may not be feasible to use the traditional approach in training ML models.

As a practical example, consider a binary GEN (BGEN) file of the UKBIOBANK data for an individual. The file has genotypic data for about 90 million SNPs. The size of data representation needed for this amount of data (using some techniques) is 9500×9500×14 (i.e., 3 channels for minor allele map, 3 channels for dominant allele map, 3 channels for allele 1 map, 3 channels for allele 2 map, and 2 positional encoding channels, as shown in the following figure). Feeding this representation directly to an ML model will be challenging due to hardware and software limitations. In addition, because each pixel in this representation matters, the ML model will have billions of parameters to digest such large inputs.

### II. Definitions

The term “initial input feature representation” may refer to a data construct that describes a fixed-size representation of an input feature, where segments of the initial input feature representation may be used to generate a multi-segment input feature representation for the noted input feature. In some embodiments, the initial input feature representation is a fixed-size representation of an input feature, the input feature comprises g feature values, each feature value corresponds to a genetic variant identifier of g genetic variants, and the initial input feature representation comprises an ordered sequence of n input feature representation values.

The term “multi-segment input feature representation” may refer to a data construct that describes patterns inferred based at least in part on segment-wise representations of input feature representation segments of an initial input feature representation associated with a corresponding input feature. In some embodiments, the multi-segment input feature representation is generated by a transformer-based machine learning model. In some embodiments, the transformer-based machine learning model is a transformer-based machine learning model (e.g., a bidirectional transformer-based machine learning model, such as a Bidirectional Encoder Representations from Transformers (BERT) machine learning model) that is configured to process m segment-wise transformer input data objects comprising a respective segment-wise transformer input data object for each of the segment-wise transformer m input feature representation segments to generate the multi-segment input feature representation, where the segment-wise transformer input data object for an input feature representation segment may be determined based at least in part on (e.g., may comprise) at least one of the following: (i) the segment-wise representation for the input feature representation segment as generated by the shared segment embedding machine learning model, (ii) the positional representation (e.g., a fixed-size positional embedding) of a segment in-sequence positional indicator for the input feature representation segment within an ordered segment sequence of the m input feature representation segments, and (iii) a chromosome representation (e.g., a fixed-size chromosome embedding) of the corresponding chromosome designation associated with the input feature representation segment.

The term “downstream prediction machine learning model” may refer to a data construct that is configured to describe parameters, hyper-parameters, and/or defined operations of a machine learning model that is configured to process a multi-segment input feature representation to generate a prediction. In some embodiments, the downstream prediction machine learning model comprises a natural language processing machine learning model. In some embodiments, the downstream prediction machine learning model is a convolutional neural network machine learning model. In some embodiments, when the multi-segment input feature representation is a one-dimensional vector, the downstream prediction machine learning model is a feedforward neural network machine learning model. In some embodiments, the downstream prediction machine learning model is trained using ground-truth historical prediction data (e.g., ground-truth historical disease labeling data associated with a group of patients). In some embodiments, inputs to the downstream prediction machine learning model comprise the multi-segment input feature representation, which may be a vector, a matrix, an image, tensor, and/or the like. In some embodiments, outputs of the downstream prediction machine learning model comprise a classification vector and/or a regression value. In some embodiments, the shared segment embedding machine learning model, the transformer-based machine learning model, and the downstream prediction machine learning model are trained in an end-to-end manner and using historical ground-truth predictions.

The term “input feature representation segment” may refer to a data construct that describes an input feature representation segment is a defined-length segment of an ordered sequence of n input feature representation values of an initial input feature representation that begins with an initial input feature representation value having an initial value in-sequence position indicator and ends with a terminal input feature representation value having a terminal value in-sequence position indicator. In some embodiments, an ordered sequence of n input feature representation values may be divided into c input feature representation super-segments, where each input feature representation segment is associated with a corresponding chromosome designation and comprises the chromosome-related subsequence for the corresponding chromosome designation. Accordingly, the ordered sequence of n input feature representation values can be divided into disjoint segments that are determined based at least in part on disjoint chromosome-related subsequences associated with the c chromosome designations. For example, where c=46, the ordered sequence of n input feature representation values may be divided into 46 input feature representation super-segments, where each input feature representation super-segment includes those input feature representation values (e.g., those genetic variant identifier values) that correspond to a particular chromosome of 46 chromosomes. Accordingly, chromosome-based demarcations can be used to create one level of segmentation across the ordered sequence of n input feature representation values. As described below, the first-level segments can then in turn be further segmented in accordance with a segmentation policy to generate second-level segments, referred to herein as input feature representation segments.

The term “segmentation policy” may refer to a data construct that defines: (i) for each chromosome designation of c chromosome designations associated with an input feature, an intra-chromosome segment count (i.e., an mᵢ value as described above), and (ii) a shared per-segment input feature representation value count that is common across m input feature representation segments generated based at least in part on the segmentation policy (where m=Σmᵢ, with i iterating over the c chromosome designations). An intra-chromosome segment count for a particular chromosome designation may describe a recommended number of input representation segments that should be generated based at least in part on the input feature representation super-segment for the chromosome designation. For example, if a particular chromosome designation is associated with an intra-chromosome segment count of 20, then the input feature representation super-segment for the particular chromosome designation should be segmentized to generate 20 input feature representation segments. In an exemplary embodiment, if the described particular chromosome designation is one of two total chromosome designations, with the other chromosome designation being associated with an intra-chromosome segment count of 30, then a total of 20+30 input feature representation segments may be generated based at least in part on the described segmentation policy. The shared per-segment input feature representation value count may describe the required/recommended number of input feature representation values from an ordered sequence of input feature representation values that should be in each input feature representation segment. For example, the shared per-segment input feature representation value count may require that each input feature representation value should include 10 input feature representation values. In some embodiments, given a segmentation policy that defines a particular intra-chromosome segment count mᵢ for an input feature representation super-segment ssᵢ as well as a particular shared per-segment input feature representation value count v, then the input feature representation values that fall within ssᵢ should be divided into mᵢ subsets (e.g., mᵢ disjoint subsets, mᵢ overlapping subsets, and/or the like), where each of the mᵢ subsets includes v of the input feature representation values that fall within ssᵢ. This may in an exemplary embodiment include, given mᵢ=2, v=20, and a total of 30 input feature representation values that fall within ssᵢ, generating a first input feature representation segment that starts with a first input feature representation value of the 30 input feature representation values that falls within ssᵢ and ends with a twentieth input feature representation value of the 30 input feature representation values that falls within ssᵢ, as well as a second input feature representation segment that starts with an eleventh input feature representation value of the 30 input feature representation values that falls within ssᵢ and ends with a thirtieth input feature representation value of the 30 input feature representation values that falls within ssᵢ.

The term “shared segment embedding machine learning model” may refer to a data construct that is configured to describe parameters, hyper-parameters, and/or defined operations of a machine learning model that is configured to, for each input feature representation segment of the m input feature representation segments: (i) generate a fixed-size data representation, and (ii) process the fixed-size data representation for the input feature representation segment using one or more machine learning layers (e.g., one or more feedforward neural network layers) to generate the segment-wise representation for the input feature representation segment. In some embodiments, each segment-wise representation generated by the shared segment embedding machine learning model is a fixed-size segment embedding for the corresponding input feature representation segment. After the m segment-wise representations are generated by the shared segment embedding machine learning model, the m segment-wise representations are processed by a transformer-based machine learning model to generate the multi-segment input feature representation. In some embodiments, the transformer-based machine learning model is a transformer-based machine learning model (e.g., a bidirectional transformer-based machine learning model, such as a Bidirectional Encoder Representations from Transformers (BERT) machine learning model) that is configured to process m segment-wise transformer input data objects comprising a respective segment-wise transformer input data object for each of the segment-wise transformer m input feature representation segments to generate the multi-segment input feature representation, where the segment-wise transformer input data object for an input feature representation segment may be determined based at least in part on (e.g., may comprise) at least one of the following: (i) the segment-wise representation for the input feature representation segment as generated by the shared segment embedding machine learning model, (ii) the positional representation (e.g., a fixed-size positional embedding) of a segment in-sequence positional indicator for the input feature representation segment within an ordered segment sequence of the m input feature representation segments, and (iii) a chromosome representation (e.g., a fixed-size chromosome embedding) of the corresponding chromosome designation associated with the input feature representation segment. In some embodiments, inputs to the shared segment embedding machine learning model include m vectors each describing a fixed-length representation of an input feature representation segment. In some embodiments, outputs of the shared segment embedding machine learning model include m segment-wise representations, where each segment-wise representation is a vector. In some embodiments, the shared segment embedding machine learning model, the transformer-based machine learning model, and the downstream prediction machine learning model are trained in an end-to-end manner and using historical ground-truth predictions.

The term “transformer-based machine learning model” may refer to a data construct that is configured to describe parameters, hyper-parameters, and/or defined operations of a transformer-based machine learning model (e.g., a bidirectional transformer-based machine learning model, such as a Bidirectional Encoder Representations from Transformers (BERT) machine learning model) that is configured to process m segment-wise transformer input data objects comprising a respective segment-wise transformer input data object for each of the segment-wise transformer m input feature representation segments to generate the multi-segment input feature representation, where the segment-wise transformer input data object for an input feature representation segment may be determined based at least in part on (e.g., may comprise) at least one of the following: (i) the segment-wise representation for the input feature representation segment as generated by the shared segment embedding machine learning model, (ii) the positional representation (e.g., a fixed-size positional embedding) of a segment in-sequence positional indicator for the input feature representation segment within an ordered segment sequence of the m input feature representation segments, and (iii) a chromosome representation (e.g., a fixed-size chromosome embedding) of the corresponding chromosome designation associated with the input feature representation segment. In some embodiments, for an ith input feature representation segment within an ordered segment sequence of m input feature representation segments that is associated with a jth chromosome designation within an ordered chromosome sequence of c chromosome designations, the input feature data object for the noted input feature representation segment may comprise the segment-wise representation for the noted input feature representation segment, a positional representation that may be a fixed-size embedding of i (i.e., of the segment in-sequence positional indicator for the noted input feature representation segment), and a chromosome representation that may be a fixed size embedding of the jth chromosome (i.e., of the corresponding chromosome designation associated with the noted input feature representation segment). The m segment-wise transformer input data objects for the m input feature representation segments may then be processed by the transformer-based machine learning model to generate the multi-segment input feature representation. In some embodiments, inputs to the transformer-based machine learning model include m segment-wise transformer input data objects, where each segment-wise transformer input data object is a vector. In some embodiments, outputs of the shared segment embedding machine learning model include a vector describing a multi-segment input feature representation. In some embodiments, the shared segment embedding machine learning model, the transformer-based machine learning model, and the downstream prediction machine learning model are trained in an end-to-end manner and using historical ground-truth predictions.

The term “input feature” may refer to a data construct that is configured to describe data pertaining to one or more individuals. In some embodiments, the input feature may comprise one or more feature values corresponding to a genetic variant identifier. Each feature value of the one or more feature values and each feature value may be associated with an input feature type designation of a plurality of input feature type designations. In some embodiments, the plurality of input feature type designations may include a DNA nucleotide, an RNA nucleotide, a minor allele frequency (MAF), a dominant allele frequency, and/or the like. In some embodiments, the one or more feature values correspond to a categorical feature type or numerical feature type. This may be dependent on which input feature type designation the feature value corresponds to. For example, a DNA nucleotide input feature type designation may be associated with feature values of a categorical feature input type, such as a feature value of “A”, representative of the DNA nucleotide adenine. As another example, a MAF input feature type designation may be associated with features value of a numerical feature type, such as a feature value of 0.2. In some embodiments, a genetic variant identifier may be associated with one or more feature values and input feature type designations. For example, a particular genetic variant identifier may be associated with the feature value ‘A’, which may be a DNA nucleotide input feature type designation, and 0.2, which may be a MAF input feature type designation. Further, these particular feature values may be associated with one another. By way of continuing example, the feature value ‘A’ associated with a DNA nucleotide input feature type designation may have an associated minor allele frequency of 0.2 as indicated by the feature value 0.2 associated with a MAF input feature type designation corresponding to the same genetic variant identifier.

The term “genetic variant identifier” may refer to a data construct that describes a particular location on genetic material. In some embodiments, the genetic variant identifier is indicative of a particular single-nucleotide polymorphism (SNP) of a particular gene. In some embodiments, the genetic variant identifier is indicative of a particular position on a chromosome (i.e. a locus) and/or the identity of the particular chromosome. In some embodiments, the genetic variant identifier is merely representative of a particular location on genetic material. For example, a genetic variant identifier “rs1” may correspond to a particular gene locus, such as, for example, the first nucleotide locus for a particular gene and/or allele. An example of a genetic variant identifier is an rsID, which is a unique label (“rs” followed by a number) given to a specific SNP.

The term “image representation” may refer to a data construct that is configured to describe, given a corresponding input feature having a plurality of input feature type designations, one or more image representations corresponding to each input feature type designations for the corresponding input feature each visually distinguishing the corresponding input feature. Furthermore, the image representation count of the one or more image representations may be based at least in part on the plurality of input feature type designations. For example, if an input feature is associated with a DNA nucleotide input feature designation type, which is a categorical feature type, an image representation for each category of the DNA nucleotide input feature designation type may be generated. As such, in this particular example, an image representation for a DNA nucleotide input feature designation type may include image representations corresponding to the DNA nucleotide categories adenine (A), thymine (T), cytosine (C), and guanine (G). As another example, if an input feature is associated with a MAF input feature designation type, which is a numerical feature type, only a single image representation may be generated.

The term “image representation region” may refer to a data construct that is configured to describe a region of an image representation for a corresponding genetic variant identifier. The number of image representation regions may be determined based at least in part on the number of genetic variant identifiers such that each genetic variant identifier corresponds to an image representation region. In some embodiments, the visual representation of the image representation region may be indicative of at least whether a particular feature value corresponding to a particular genetic variant identifier is present or absent in the input feature.

The term “positional encoding map” may refer to a data construct that is configured, within a plurality of position encoding maps comprising a plurality of positional encoding map region sets, to describe data associated with a particular genetic variant identifier. A positional encoding map may be comprised of positional encoding map regions each corresponding to a genetic variant identifier. Each region of a positional encoding map may correspond to an identifier value. For example, the first positional encoding map region may comprise an identifier value of ‘1’, the second positional encoding map region may comprise an identifier value of ‘2’, etc. A positional encoding map set may comprise each positional encoding map region corresponding to the same genetic variant identifier across the plurality of positional encoding maps. For example, if the plurality of positional encoding maps comprise two positional encoding maps, and the positional encoding map regions corresponding to the first genetic variant identifier in both positional encoding maps comprise an identifier value of ‘1’, the positional encoding map region set for the first genetic variant identifier may comprise the identifier values ‘1,1’.

The term “first allele image representation” may refer to a data construct that is configured to describe a representation of a genetic sequence associated with an individual as indicated by feature values of an input feature associated with an individual. In some embodiments, the genetic sequence corresponds to one or more particular genes and/or alleles for a first chromosome and/or first set of chromosomes of the individual.

The term “second allele image representation” may refer to a data construct that is configured to describe a representation of a genetic sequence associated with an individual as indicated by feature values of an input feature associated with an individual. In some embodiments, the genetic sequence corresponds to a particular gene and/or allele. In some embodiments, the genetic sequence corresponds to one or more particular genes and/or alleles for a second chromosome and/or second set of chromosomes of the individual. In some embodiments, the individual associated with the second allele image is the same individual associated with the first allele image representation. In some embodiments, the individual associated with the second allele image is a different individual than the individual associated with the first allele image representation.

The term “dominant allele image representation” may refer to a data construct that is configured to describe a representation of a genetic sequence associated with a dominant genetic sequence for a particular genetic sequence as indicated by feature values of an input feature. In some embodiments, the genetic sequence corresponds to a particular gene and/or allele. In some embodiments, the dominant genetic sequence is the genetic sequence most common in a population.

The term “minor allele image representation” m may refer to a data construct that is configured to describe a representation of a genetic sequence associated with a minor genetic sequence for a particular genetic sequence as indicated by feature values of an input feature. In some embodiments, the genetic sequence corresponds to a particular gene and/or allele. In some embodiments, the minor genetic sequence is the genetic sequence associated with a second most common genetic sequence in a population. In some embodiments, the minor genetic sequence is a genetic sequence associated other than the most common genetic sequence in a population.

The term “differential image representation” may refer to a data construct that is configured to describe an image representation of a difference between a first image representation and a second image representation. In some embodiments, the differential image representation may be generated based at least in part on a comparison between a first allele image representation or second allele image representation and dominant allele image representation or minor allele image representation using one or more mathematical and/or logical operators. In some embodiments, the differential image representation may be generated based at least in part on a comparison between the first allele image representation and a second allele image representation corresponding to one or more individuals using one or more mathematical and/or logical operators. For example, if a first allele image representation indicates a feature value of ‘A’ in the image region corresponding to the first genetic variant identifier and a second allele image representation indicates a feature value of ‘A’ in the image region corresponding to the first genetic variant identifier, the image region of the differential image representation corresponding to the first genetic variant identifier may be indicative of a match between the first allele image representation and second allele image representation. As another example, if a first allele image representation indicates a feature value of ‘A’ in the image region corresponding to the second genetic variant identifier and a second allele image representation indicates a feature value of ‘C’ in the image region corresponding to the second genetic variant identifier, the image region of the differential image representation corresponding to the second genetic variant identifier may be indicative of a difference between the first allele image representation and second allele image representation. A match and/or difference in the image region for the differential image representation may be indicated in a variety of ways including using numerical values, colors, and/or the like. For example, a match between image regions in the first image representation and second image representation may be indicated by an image region value of ‘1’ and a non-match between image regions in the first image representation and second image representation may be indicated by an image region value of ‘0’. As another example, a match between image regions in the first image representation and second image representation may be indicated by a white color in the corresponding image region while a non-match between image regions in the first image representation and second image representation may be indicated by a black color in the corresponding image region.

The term “zygosity image representation” may refer to a data construct that is configured to describe a representation of a zygosity associated with an individual based at least in part on an associated first allele image representation and a second allele image representation for the individual, a dominant allele representation, and a minor allele representation for a genetic sequence (e.g. gene, allele, chromosome, etc.). In some embodiments, the zygosity image representation may be generated based at least in part on a comparison between the first allele image representation and a second allele image representation using one or more mathematical and/or logical operators, similar to the differential image representation. Further, the zygosity image representation may be generated based at least in part on a comparison between the first allele image representation, second allele image representation, dominant allele representation, and minor allele representation using one or more mathematical and/or logical operators. For example, if an individual is associated with a first allele image representation indicating a feature value of ‘A’ in the image region corresponding to the second genetic variant identifier and a second allele image representation indicates a feature value of ‘C’ in the image region corresponding to the second genetic variant identifier, the feature value for the second genetic variant identifier is determined to be heterozygous. As another example, if an individual is associated with a first allele image representation indicating a feature value of ‘A’ in the image region corresponding to the first genetic variant identifier and a second allele image representation indicates a feature value of ‘A’ in the image region corresponding to the first genetic variant identifier, the feature value for the first genetic variant is determined to be homozygous. Further, the homozygous feature value of ‘A’ may be compared to the feature values corresponding to the first genetic variant identifier in the dominant allele image representation and/or minor allele image representation. If the homozygous feature value matches the feature value in the dominant allele image representation, the feature value is determined to be homozygous with a dominant allele. If the homozygous feature value matches the feature value in the minor allele image representation, the feature value is determined to be homozygous with a minor allele. A heterozygous, homozygous with a dominant allele, homozygous with a minor allele, etc. may be indicated in a variety of ways including using values corresponding to each category, colors corresponding to each category, etc. For example, an image region determined to be heterozygous may be associated with a value of ‘0’, an image region determined to be homozygous with a dominant allele may be associated with a value of ‘1’, and an image region determined to be homozygous with a dominant allele may be associated with a value of ‘2’. As another example, an image region determined to be heterozygous may be associated with a green color, an image region determined to be homozygous with a dominant allele may be associated with a red color, and an image region determined to be homozygous with a dominant allele may be associated with a blue color.

The term “intensity image representation” may refer to a data construct that is configured to describe feature values of an input feature type designation using one or more assigned intensity values for each input feature type designation. In some embodiments, input feature type designations associated with feature values corresponding to a categorical feature type may have an intensity value assigned for each category of the input feature type designation. For example, a DNA nucleotide input feature type designation may be associated with categories ‘A’, ‘C’, ‘T’, ‘G’, and missing (corresponding to adenine, cytosine, thymine, and guanine, respectively) may be assigned intensity values 1, 0.75, 0.5, 0.25, and 0. Additionally or alternatively, the categories ‘A’, ‘C’, ‘T’, ‘G’, and missing may be assigned intensity values corresponding to the colors red, green, blue, white, and black, respectively. In some embodiments, input feature type designations associated with feature values corresponding to a numeric feature type may have an intensity value based at least in part on the numeric value of the feature value. For example, a MAF input feature type designation may be associated with a numeric value between 0 and 1. As such, a feature value of ‘0.3’ for an MAF input feature type designation may be associated with an intensity value of 0.3. In some embodiments, intensity value for a feature value corresponding to a numeric input feature type may be rounded to the nearest integer or decimal place of interest. For example, a feature value of 0.312 for an MAF input feature type designation may be associated with an intensity value of 0.3.

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises a combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

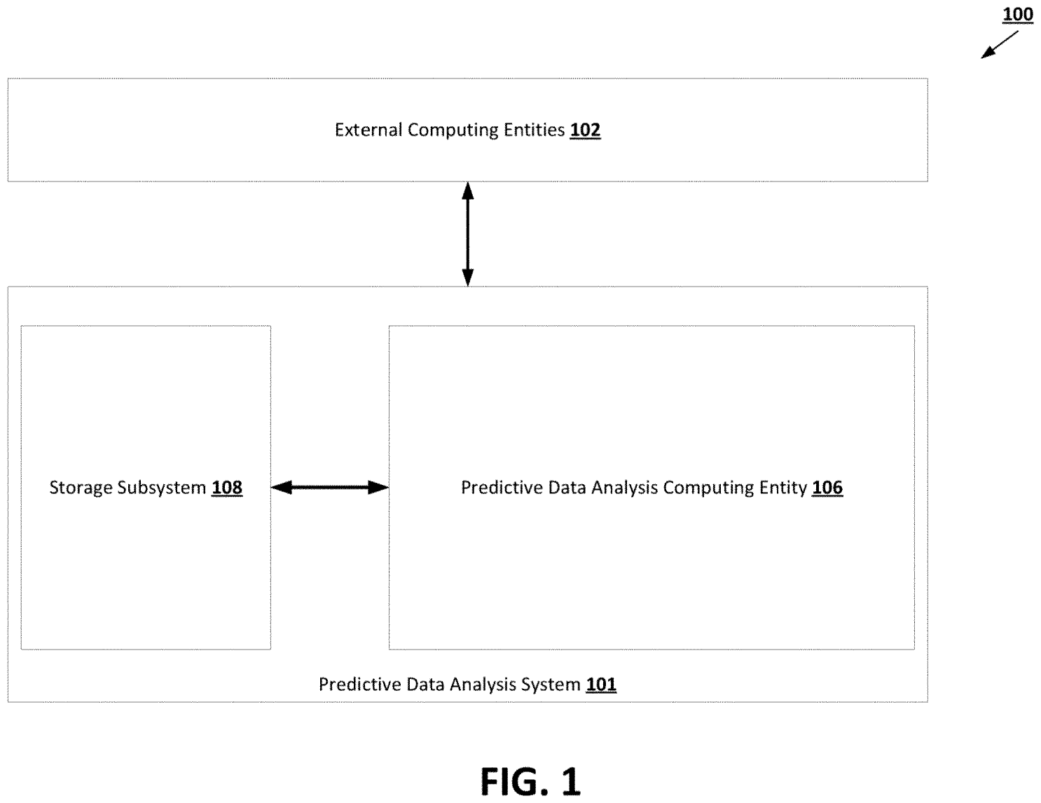

FIG. 1 is a schematic diagram of an example architecture 100 for performing health-related predictive data analysis. The architecture 100 includes a predictive data analysis system 101 configured to receive health-related predictive data analysis requests from external computing entities 102, process the predictive data analysis requests to generate health-related risk predictions, provide the generated health-related risk predictions to the external computing entities 102, and automatically perform prediction-based actions based at least in part on the generated health-related risk predictions. Examples of health-related predictions include genetic risk predictions, polygenic risk predictions, medical risk predictions, clinical risk predictions, behavioral risk predictions, and/or the like.

In some embodiments, predictive data analysis system 101 may communicate with at least one of the external computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software, and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive health-related predictive data analysis requests from one or more external computing entities 102, process the predictive data analysis requests to generate the polygenic risk score predictions corresponding to the predictive data analysis requests, provide the generated polygenic risk score predictions to the external computing entities 102, and automatically perform prediction-based actions based at least in part on the generated polygenic risk score predictions.

The storage subsystem 108 may be configured to store input data used by the predictive data analysis computing entity 106 to perform health-related predictive data analysis, as well as model definition data used by the predictive data analysis computing entity 106 to perform various health-related predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media, including but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

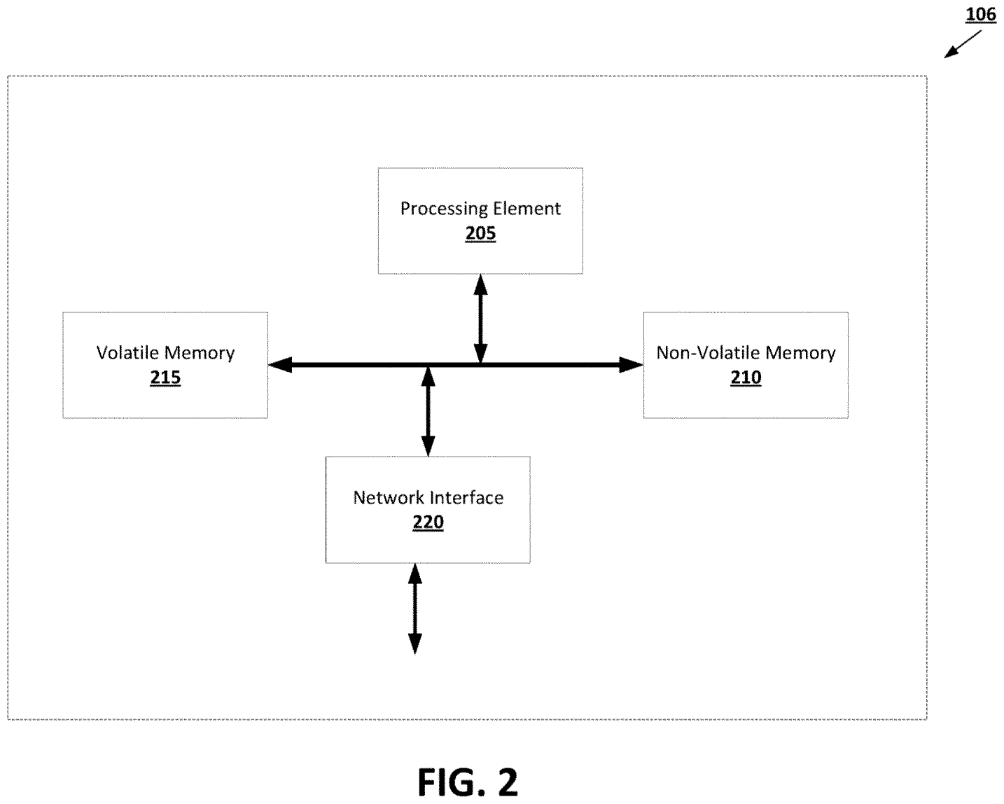

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include or be in communication with one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

In one embodiment, the predictive data analysis computing entity 106 may further include or be in communication with volatile media (also referred to as volatile storage, memory, memory storage, memory circuitry, and/or similar terms used herein interchangeably). In one embodiment, the volatile storage or memory may also include one or more volatile storage or memory media 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include or be in communication with one or more input elements, such as a keyboard input, a mouse input, a touch screen/display input, motion input, movement input, audio input, pointing device input, joystick input, keypad input, and/or the like. The predictive data analysis computing entity 106 may also include or be in communication with one or more output elements (not shown), such as audio output, video output, screen/display output, motion output, movement output, and/or the like.

### Exemplary External Computing Entity

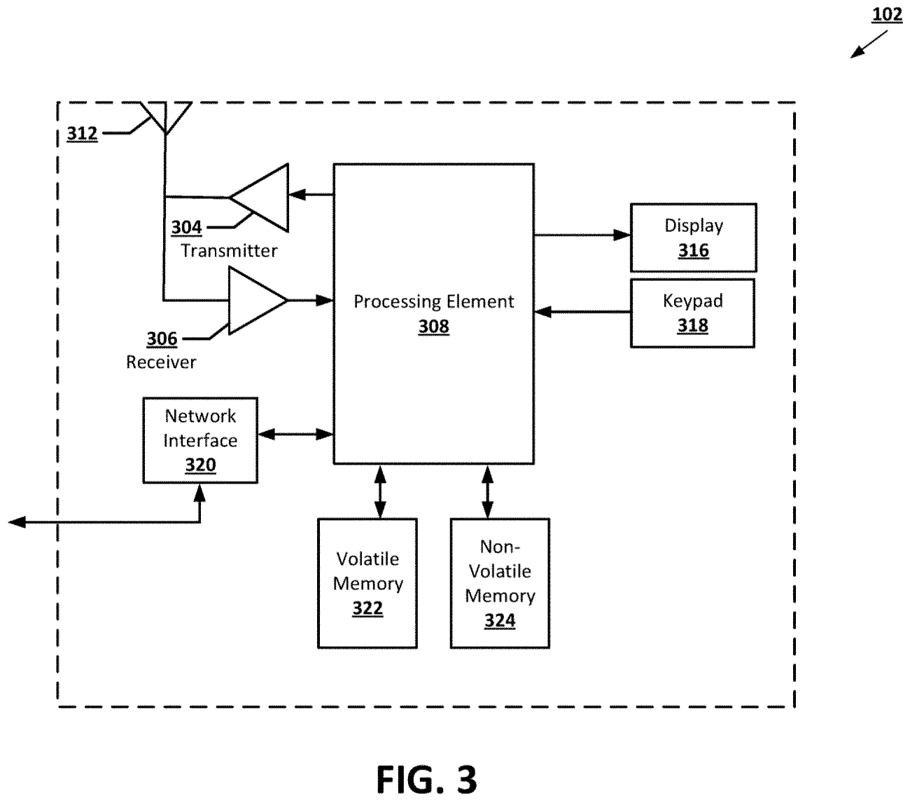

FIG. 3 provides an illustrative schematic representative of an external computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. External computing entities 102 can be operated by various parties. As shown in FIG. 3, the external computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the external computing entity 102 may be capable of operating with one or more air interface standards, communication protocols, modulation types, and access types. More particularly, the external computing entity 102 may operate in accordance with any number of wireless communication standards and protocols, such as those described above with regard to the predictive data analysis computing entity 106. In a particular embodiment, the external computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 may operate in accordance with multiple wired communication standards and protocols, such as those described above with regard to the predictive data analysis computing entity 106 via a network interface 320.

Via these communication standards and protocols, the external computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), and operating system.

According to one embodiment, the external computing entity 102 may include location determining aspects, devices, modules, functionalities, and/or similar words used herein interchangeably. For example, the external computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the external computing entity's 102 position in connection with a variety of other systems, including cellular towers, Wi-Fi access points, and/or the like. Similarly, the external computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops), and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The external computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the external computing entity 102 to interact with and/or cause display of information/data from the predictive data analysis computing entity 106, as described herein. The user input interface can comprise any of a number of devices or interfaces allowing the external computing entity 102 to receive data, such as a keypad 318 (hard or soft), a touch display, voice/speech or motion interfaces, or another input device. In embodiments including a keypad 318, the keypad 318 can include (or cause display of) the conventional numeric (0-9) and related keys (#, *), and other keys used for operating the external computing entity 102 and may include a full set of alphabetic keys or a set of keys that may be activated to provide a full set of alphanumeric keys. In addition to providing input, the user input interface can be used, for example, to activate or deactivate certain functions, such as screen savers and/or sleep modes.

The external computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the external computing entity 102. As indicated, this may include a user application that is resident on the entity or accessible through a browser or other user interface for communicating with the predictive data analysis computing entity 106 and/or various other computing entities.

In another embodiment, the external computing entity 102 may include one or more components or functionalities that are the same or similar to those of the predictive data analysis computing entity 106, as described in greater detail above. As will be recognized, these architectures and descriptions are provided for exemplary purposes only and are not limiting to the various embodiments.

In various embodiments, the external computing entity 102 may be embodied as an artificial intelligence (AI) computing entity, such as an Amazon Echo, Amazon Echo Dot, Amazon Show, Google Home, and/or the like. Accordingly, the external computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

As described below, various embodiments of the present invention address technical challenges related to efficiently performing machine learning tasks on large datasets and/or on data-intensive datasets. As described below, in various embodiments of the present invention, a large and/or data-intensive dataset is converted into input feature representation super-segments and input feature representation segments, where the input feature representation super-segments are mapped to sentences and input feature representation segments are mapped to words. Then, segment-wise representations for input feature representation segments are provided to a transformer-based language model in accordance with the sentence-word hierarchy described above to generate multi-segment input feature representations that can then be used to perform efficient and effective predictive data analysis operations. This highlights a major technical advantage of the noted embodiments of the present invention: instead of processing an initial input feature representation as a whole, the noted embodiments of the present invention first generate m input feature representation segments of the initial input feature representation, and then process the m input feature representation segments using efficient and effective transformer-based language models. As a result, instead of performing the often excessively large computational task of processing the initial input feature representation as a whole and using an excessively large amount of computational resources and a large amount of processing time, various embodiments of the present invention divide the noted computational task into smaller computational sub-tasks that can be more manageably executed using transformer-based language models and by utilizing the sentence-word hierarchy described above. In this way, various embodiments of the present invention enable faster and less-resource-intensive processing of large machine learning tasks and/or data-intensive machine learning tasks by hierarchically segmenting input spaces and using the noted hierarchical segmentations to enable transformer-based encoding of the noted input spaces.

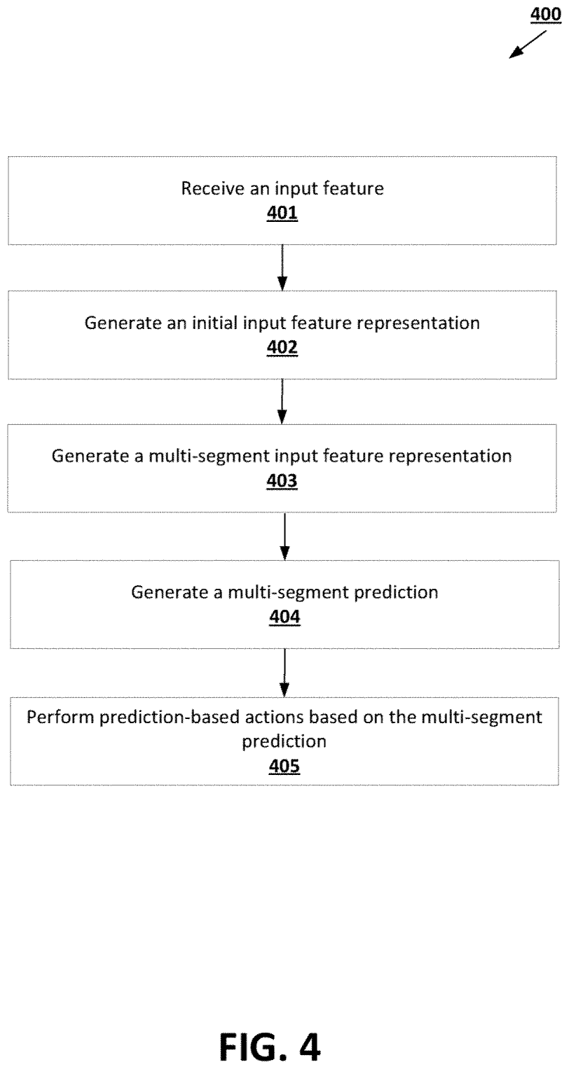

FIG. 4 is a flowchart diagram of an example process 400 for generating a multi-segment prediction for an input feature. Via the various steps/operations of the process 400, the predictive data analysis computing entity 106 can generate multi-segment predictions using at least one of segment-wise feature processing machine learning models or a multi-segment representation machine learning model.

At step/operation 401, the predictive data analysis computing entity 106 receives an input feature. Examples of an input feature include structured text input features, including feature data associated with a predictive entity. For example, the input feature may describe data pertaining to one or more individuals. In some embodiments, the input feature may comprise one or more (e.g., a defined number of, such as g) input feature values corresponding to a genetic variant identifier. Each feature value of the one or more feature values may be associated with an input feature type designation of a plurality of input feature type designations. In some embodiments, the plurality of input feature type designations may include a DNA nucleotide, an RNA nucleotide, a minor allele frequency (MAF), a dominant allele frequency, and/or the like.

In some embodiments, the feature values correspond to a categorical feature type or numerical feature type. This may be dependent on which input feature type designation the feature value corresponds to. For example, a DNA nucleotide input feature type designation may be associated with feature values of a categorical feature input type, such as a feature value of “A”, representative of the DNA nucleotide adenine. As another example, a MAF input feature type designation may be associated with feature values of a numerical feature type, such as a feature value of 0.2. In some embodiments, a genetic variant identifier may be associated with one or more feature values and input feature type designations. For example, a particular genetic variant identifier may be associated with the feature value ‘A’, which may be a DNA nucleotide input feature type designation, and 0.2, which may be a MAF input feature type designation. Further, these particular feature values may be associated with one another. By way of continuing example, the feature value ‘A’ associated with a DNA nucleotide input feature type designation may have an associated minor allele frequency of 0.2 as indicated by the feature value 0.2 associated with a MAF input feature type designation corresponding to the same genetic variant identifier.

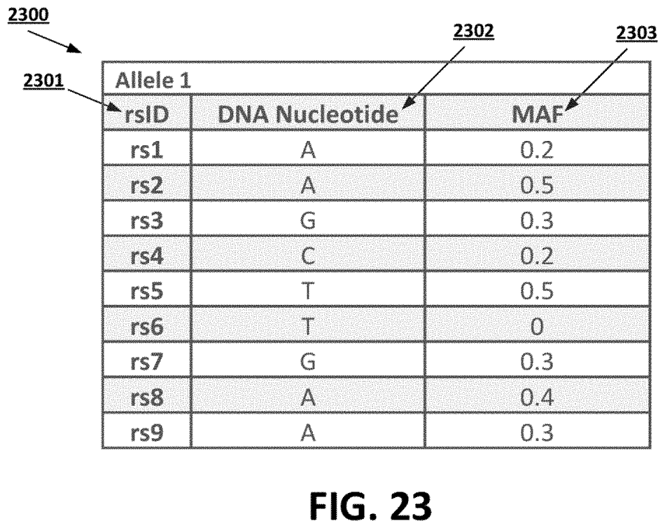

An operational example of an input feature 2300 is depicted in FIG. 23. By way of example, an input feature may comprise feature values “A”, “A”, “G”, “C”, “T”, “T”, “G” , “A”, and “A” corresponding to the input feature type designation DNA nucleotide 2302 and feature values “0.2”, “0.5”, “0.3”, “0.2”, “0.5”, “0”, “0.3”, “0.4”, “0.3” corresponding to the input feature type designation MAF 2303. Additionally, each feature value of the input feature may correspond to a genetic variant identifier 2301.

In some embodiments, the predictive data analysis computing entity 106 may identify one or more feature values from an input feature structured as a text sequence. The predictive data analysis computing entity 106 may identify the one or more feature values in a variety of ways, such as by using a delimiter. For example, the boundary between separate feature values of the input feature may be indicated by a predefined character, such as a comma, semicolon, quotes, braces, pipes, slashes, etc. In the above example, a boundary between feature values may be indicated by a comma such that structured text sequence “A, A, G, C, T, T, G, A, A” corresponds to feature values “A”, “A”, “G”, “C”, “T”, “T”, “G” , “A”, and “A”. Additionally or alternatively, in some embodiments, the predictive data analysis computing entity 106 may identify one or more feature values based at least in part on the input feature type designation of a structured text sequence. For example, an input feature comprising the structured text sequence “AAGCTTGAA” may correspond to a DNA nucleotide input feature type designation. A predictive data analysis computing entity 106 may be configured to automatically identify each character comprising the structured text sequence associated with a DNA nucleotide input feature type designation such that the predictive data analysis computing entity 106 may automatically identify the feature values “A”, “A”, “G”, “C”, “T”, “T”, “G” , “A”, and “A” without the use of delimiters.

At step/operation 402, the predictive data analysis computing entity 106 generates an initial input feature representation of the input feature. Exemplary techniques for generating an input feature representation for an input feature are described in Subsection A of the present Section IV. However, a person of ordinary skill in the relevant technology will recognize that other techniques for generating fixed-size representations of input features (e.g., fixed-size image representation of input features) may be used to generate initial input feature representations in accordance with various embodiments of the present invention. In some embodiments, the initial input feature representation is a fixed-size representation of an input feature, the input feature comprises g feature values, each feature value corresponds to a genetic variant identifier of g genetic variants, and the initial input feature representation comprises an ordered sequence of n input feature representation values.

At step/operation 403, the predictive data analysis computing entity 106 generates a multi-segment input feature representation of the input feature based at least in part on the initial input feature representation of the input feature. Exemplary techniques for generating multi-segment input feature representations are described in Subsection B of the present Section IV. However, a person of ordinary skill in the relevant technology will recognize that other techniques for generating multi-segment input feature representations based at least in part on initial input feature representations may be utilized in accordance with various embodiments of the present invention.

At step/operation 404, the predictive data analysis computing entity 106 generates, based at least in part on the multi-segment input feature representation and using a downstream prediction machine learning model, the multi-segment prediction. In some embodiments, when the multi-segment input feature representation is an image representation, then the downstream prediction machine learning model is a convolutional neural network machine learning model. In some embodiments, when the multi-segment input feature representation is a one-dimensional vector, the downstream prediction machine learning model is a feedforward neural network machine learning model.

At step/operation 405, the predictive analysis engine 112 performs a prediction-based action based at least in part on the predictions generated in step/operation 404. Examples of prediction-based actions include transmission of communications, activation of alerts, automatic scheduling of appointments, and/or the like. As a further example, the predictive analysis engine 112 may determine a polygenic risk score (PRS) for one or more diseases for one or more individuals based at least in part on the predictions generated in step/operation 404.

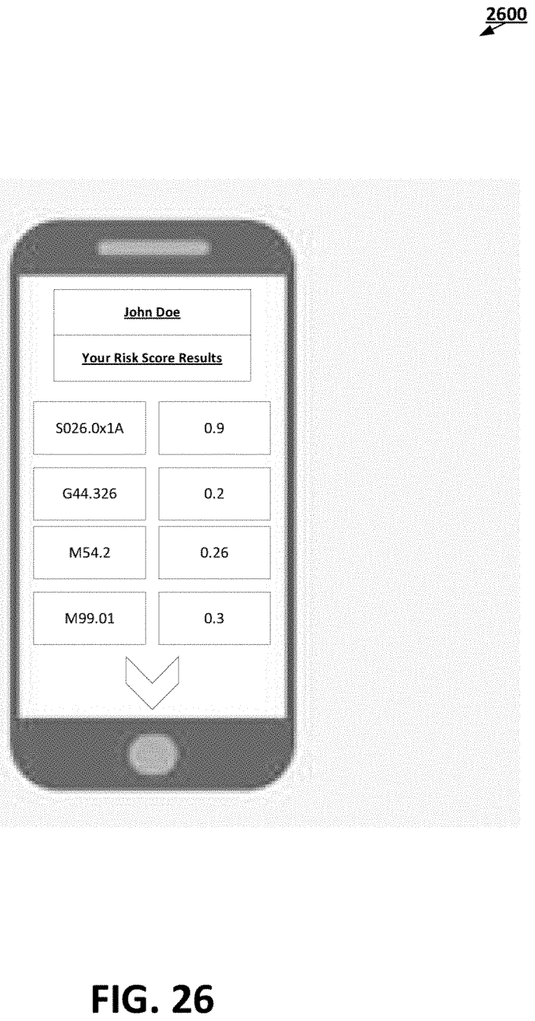

Other prediction-based actions include displaying a user interface that displays health-related risk predictions (e.g., at least one of epistatic polygenic risk scores, epistatic interaction scores, and base polygenic risk scores) for a target individual with respect to a set of conditions. For example, as depicted in FIG. 26, the predictive output user interface 2600 depicts the health-related risk prediction for a target individual with respect to four target conditions each identified by the International Statistical Classification of Diseases and Related Health Problems (ICD) code of the noted four target conditions.

Other examples of prediction-based actions include one or more optimized scheduling operations for medical appointments scheduled when health-related risk predictions indicate a need for scheduling medical appointment (e.g., a disease score described by the predictive output for a rare disease predictive task satisfies a disease score threshold). Examples of optimized scheduling operations include automatically scheduling appointments and automatically generating/triggering appointment notifications. In some embodiments, performing optimized scheduling operations includes automated system load balancing operations and/or automated staff allocation management operations. For example, an optimized appointment prediction system may automatically and/or dynamically process a plurality of event data objects in order to generate optimized appointment predictions for a plurality of patients requiring appointments with one or more providers. As another example, the optimized appointment prediction system may account for patient and/or provider availability on particular days and at particular times. In another example, the optimized appointment prediction system may reassign patients on a schedule in response to receiving real-time information, such as an instance in which a provider is suddenly unavailable due to an emergency or unplanned event/occurrence. Additionally, in some embodiments, the optimized appointment prediction system may be used in conjunction with an Electronic Health Record (EHR) system that is accessible by patients and providers to recommend a particular provider and/or automatically schedule an appointment with a particular provider in response to a request initiated by a patient. In some embodiments, the optimized appointment prediction system may aggregate a plurality of requests (e.g., from patients and/or providers) and generate one or more schedules in response to determining that a threshold number of requests have been received.

In another example, performing optimized scheduling operations includes providing additional appointment information/data (e.g., travel information, medication information, provider information, patient information and/or the like). By way of example, the optimized appointment prediction system may automatically provide pre-generated travel directions for navigating to and returning from an appointment location based at least in part on expected travel patterns at an expected end-time of the appointment. In some embodiments, the pre-generated travel directions may be based at least in part on analysis of travel patterns associated with a plurality of patients that have had appointments with a particular provider and/or at a particular location within a predefined time period.

In some embodiments, performing the optimized scheduling operations includes performing system load balancing operations for a medical record keeping system. For example, upon detecting that a medical appointment takes x minutes, computing resources of a medical record keeping system may be reassigned to ensure that adequate resources are available in order to facilitate medical record keeping, as well as retrieval of data during the medical visit. In some embodiments, performing the optimized scheduling operations may detect that an appointment ends at a particular time, and provide optimal driving directions for a post-appointment trip given expected traffic conditions at the particular time.

A. Generating Initial Input Feature Representations

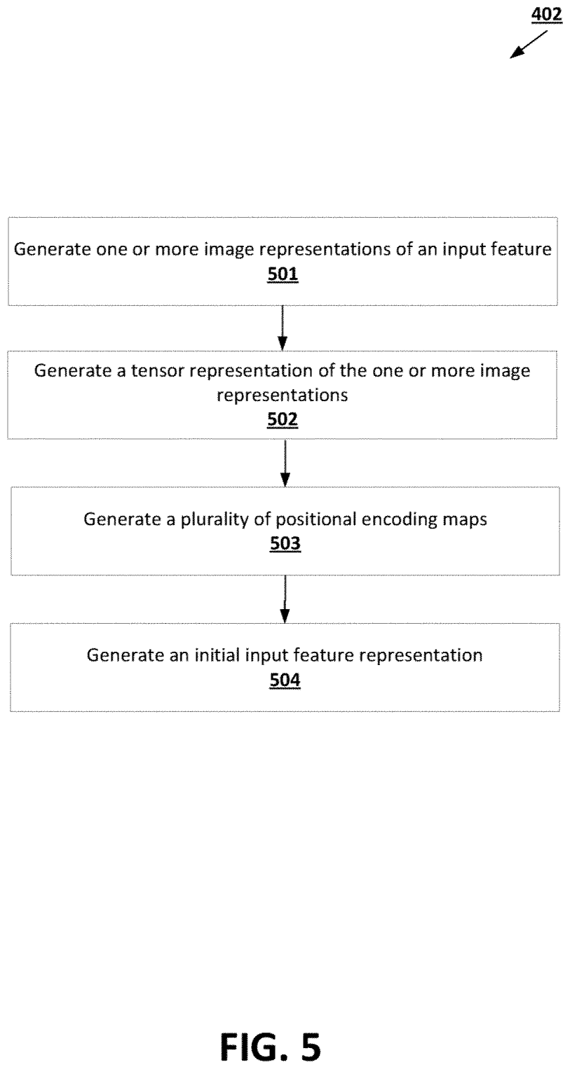

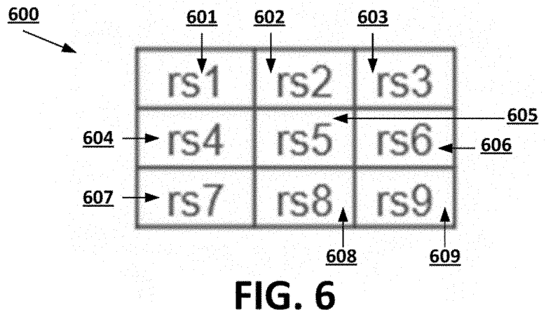

In some embodiments, step/operation 402 may be performed in accordance with the process that is depicted in FIG. 5. The process that is depicted in FIG. 5 begins at step/operation 501 when the predictive data analysis computing entity 106 generates one or more image representations based at least in part on the input feature obtained/received in step/operation 501. In some embodiments, to generate the one or more image representations based at least in part on the input feature, a feature extraction engine of the predictive data analysis computing entity 106 retrieves configuration data for a particular image-based processing routine from model definition data stored in the storage subsystem 108. Examples of the particular-image-based processing routines are discussed below with reference to FIGS. 6-23. However, one of ordinary skill in the art will recognize that the predictive data analysis computing entity 106 may generate the one or more images by applying any suitable technique for transforming the input feature into the one or more images. In some embodiments, the predictive data analysis computing entity 106 selects a suitable image-based processing routine for the input feature given the one or more properties of the input feature (e.g., inclusion of input feature type designations for the input feature, an indication of feature values pertaining to one or more individuals, and/or the like). In some embodiments, the predictive data analysis computing entity 106 may select a suitable image-based processing routine for the input feature based at least in part on a user specified preference. In some embodiments, the user specified preference may be indicated in the input feature.

An operational example of generating an image representation 600 is depicted in FIG. 6. As previously described, each feature value of the input feature may correspond to a genetic variant identifier. As such, the predictive data analysis computing entity 106 may determine an image representation 600 comprising one or more image regions 601-609. Each image region 601-609 may correspond to a genetic variant identifier, as described by the input feature received in step/operation 501. For example, if the input feature comprises feature values corresponding to nine genetic variant identifiers, the predictive data analysis computing entity 106 may determine an image representation 600 comprising nine image regions. The image representation 600 may then be used when generating the one or more image representations. Each of the one or more image regions may comprise one or more pixels and be associated with a length dimension and width dimension. In some embodiments, each of the one or more image regions may comprise the same number of pixels. In some embodiments, each of the one or more image regions may comprise the same length dimension and width dimension.

In some embodiments, the image representation 600 is associated with a length dimension and width dimensions based at least in part on the length dimension and width dimension of each of the one or more image regions. In some embodiments, the arrangement of the one or more image regions comprising the image representation 600 may be determined by the predictive data analysis computing entity 106. In some embodiments, the predictive data analysis computing entity 106 may determine the arrangement of the one or more image regions comprising the image representation 600 based at least in part on the length dimension and width dimension of the one or more image regions. In some embodiments, the predictive data analysis computing entity 106 may determine the arrangement of the one or more image regions comprising the image representation 600 such that values of the length dimension and width dimension of the image representation 600 are as close as possible. For example, the predictive data analysis computing entity 106 may determine a length dimension value of 3 and width dimension value of 3 for an image representation 600 comprising nine image regions each comprising a length dimension of 1 pixel and a width dimension of 1 pixel. As such, the image representation configuration may be square or rectangular in shape.

In some embodiments, the predictive data analysis computing entity 106 may determine to order the image regions each corresponding to a genetic variant identifier in order of the one or more genetic variant identifier such that each image region corresponding to a genetic variant identifier is adjacent to the image region corresponding to the next sequential genetic variant identifier. For example, as shown in FIG. 6, an image region 601 corresponding to a genetic identifier rs1 is adjacent to an image region 602 corresponding to a genetic identifier rs2. As another example, an image region 601 corresponding to a genetic identifier rs1 may also be adjacent to an image region 604 corresponding to a genetic identifier rs2 (not shown in FIG. 6).

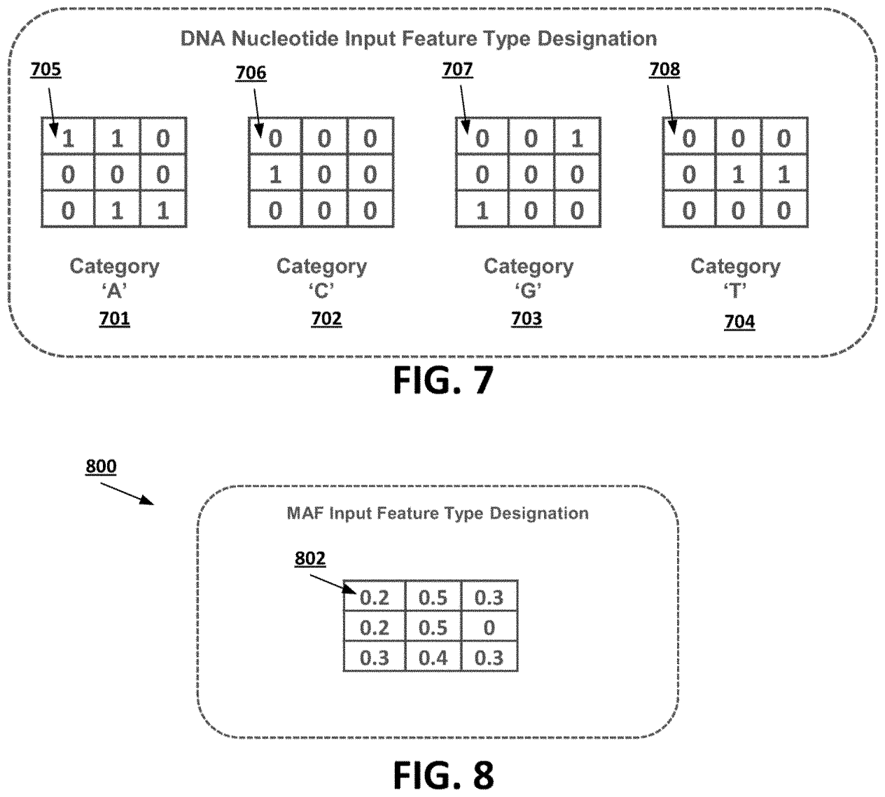

Another operational example of four image representations 701-704 for a categorical feature type is depicted in FIG. 7. In this particular example, a DNA nucleotide input feature type designation is shown, wherein the DNA nucleotide input feature type designation is a categorical input feature type. In particular, the DNA nucleotide input feature type designation is associated with 4 categories: ‘A’, ‘C’, ‘G’, and ‘T’. Each category of the DNA nucleotide input feature type designation has a corresponding image representation 701-704. The image representation for each category is based at least in part on the image representation configuration depicted in FIG. 6 and the feature values of the input feature. For example, if the feature value for the first genetic identifier rs1 is ‘A’, the value of the image region corresponding to the first genetic identifier rs1 for the image representation for the category ‘A’ may be affirmative of the value ‘A’. This may be communicated in a variety of ways, such as by a binary system where 1 indicates the presence of the corresponding category and where 0 indicates the absence of the corresponding category for each genetic variant identifier. In this instance, since the feature value for the first genetic identifier rs1 is ‘A’, the image region 705 corresponding to the first genetic identifier for the category ‘A’ is assigned a value of 1 and the image regions 706-708 corresponding to the first genetic identifier for the categories ‘C’, ‘G’, and ‘T’ is assigned a value of 0.

Another operational example of generating an image representation 800 for a numerical feature type is depicted in FIG. 8. In this particular example, a MAF input feature type designation is shown, wherein the MAF input feature type designation is a numerical input feature type. In contrast to categorical input feature types, numerical input feature types may only be associated with one image representation. The image representation 800 is based at least in part on the image representation configuration depicted in FIG. 6 and the feature values of the input feature. For example, if the feature value for the first genetic identifier rs1 is ‘0.2’, the value of the image region corresponding to the first genetic identifier rs1 for the image representation may be ‘0.2’. In this instance, since the feature value for the first genetic identifier rs1 is ‘0.2’, the image region 802 is assigned a value of ‘0.2’.

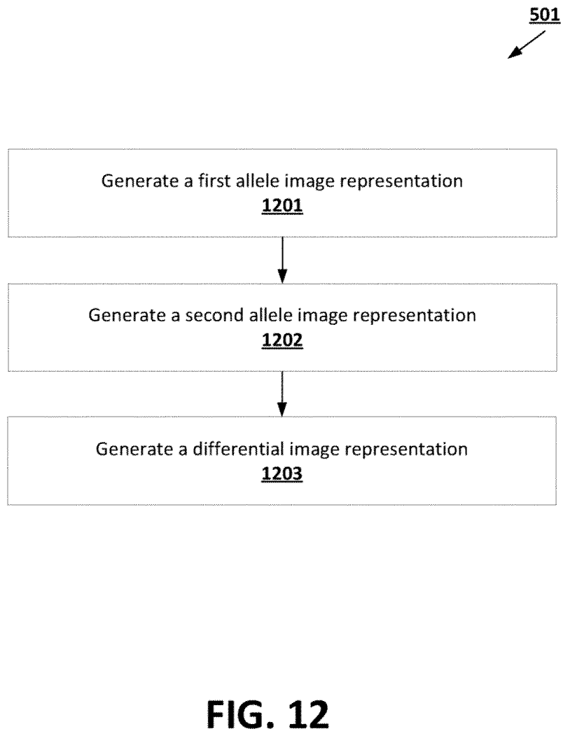

In some embodiments, step/operation 501 may be performed in accordance with the various steps/operations of the process that depicted in FIG. 12, which is a flowchart diagram of an example process for generating a differential image representation. The process that is depicted in FIG. 12 begins at step/operation 1201, when the predictive data analysis computing entity 106 generates a first allele image representation. In some embodiments, an input feature may describe a representation of a genetic sequence associated with an individual, as indicated by feature values of an input feature associated with an individual. In some embodiments, the genetic sequence corresponds to one or more particular genes and/or alleles for a first chromosome and/or first set of chromosomes of the individual.

At step/operation 1202, the predictive data analysis computing entity 106 generates a second allele image representation. In some embodiments, an input feature may describe a representation of a genetic sequence associated with an individual, as indicated by feature values of an input feature associated with an individual. In some embodiments, the genetic sequence corresponds to one or more particular genes and/or alleles for a second chromosome and/or a second set of chromosomes of the individual. In some embodiments, the individual associated with the second allele image is the same individual associated with the first allele image representation. In some embodiments, the individual associated with the second allele image is a different individual than the individual associated with the first allele image representation.

At step/operation 1203, the predictive data analysis computing entity 106 generates a differential image representation. In some embodiments, the differential image representation may be generated based at least in part on a comparison between a first allele image representation or a second allele image representation and a dominant allele image representation or a minor allele image representation using one or more mathematical and/or logical operators. In some embodiments, the differential image representation may be generated based at least in part on a comparison between the first allele image representation and a second allele image representation corresponding to one or more individuals using one or more mathematical and/or logical operators. For example, if a first allele image representation indicates a feature value of ‘A’ in the image region corresponding to the first genetic variant identifier and a second allele image representation indicates a feature value of ‘A’ in the image region corresponding to the first genetic variant identifier, the image region of the differential image representation corresponding to the first genetic variant identifier may be indicative of a match between the first allele image representation and the second allele image representation.

As another example, if a first allele image representation indicates a feature value of ‘A’ in the image region corresponding to the second genetic variant identifier and a second allele image representation indicates a feature value of ‘C’ in the image region corresponding to the second genetic variant identifier, the image region of the differential image representation corresponding to the second genetic variant identifier may be indicative of a difference between the first allele image representation and the second allele image representation. A match and/or difference in the image region for the differential image representation may be indicated in a variety of ways, including using numerical values, colors, and/or the like. For example, a match between image regions in the first image representation and the second image representation may be indicated by an image region value of ‘1’ and a non-match between image regions in the first image representation and the second image representation may be indicated by an image region value of ‘0’.

As another example, a match between image regions in the first image representation and the second image representation may be indicated by a white color in the corresponding image region while a non-match between image regions in the first image representation and second image representation may be indicated by a black color in the corresponding image region.

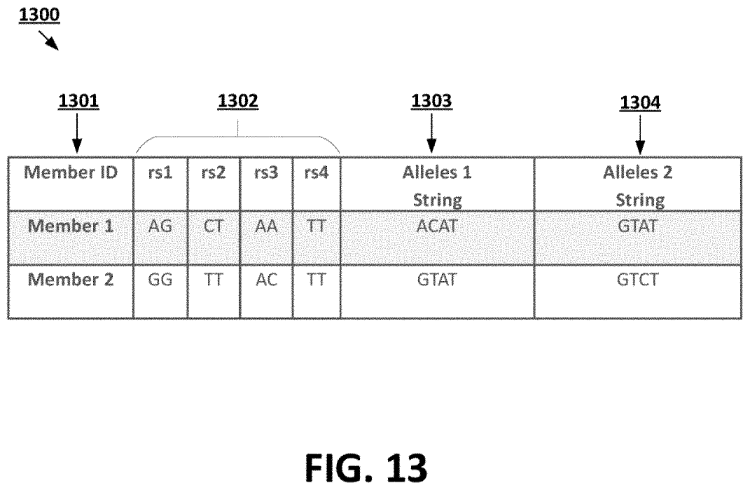

An operational example of an input feature 1300 that may be used to generate a differential image representation is depicted in FIG. 13. The input feature 1300 may comprise one or more feature values corresponding to one or more genetic variants 1302 for one or more individuals 1301. Based at least in part on these one or more feature values provided for the one or more individuals, a first allele value 1303 and a second allele value 1304 may be determined. For example, an individual with the feature values ‘AG’ for the genetic variant identifier rs1 may correspond to a value ‘A’ for the first allele value corresponding to the genetic variant identifier rs1 and a value ‘G’ for the second allele value corresponding to the genetic variant identifier rs1.

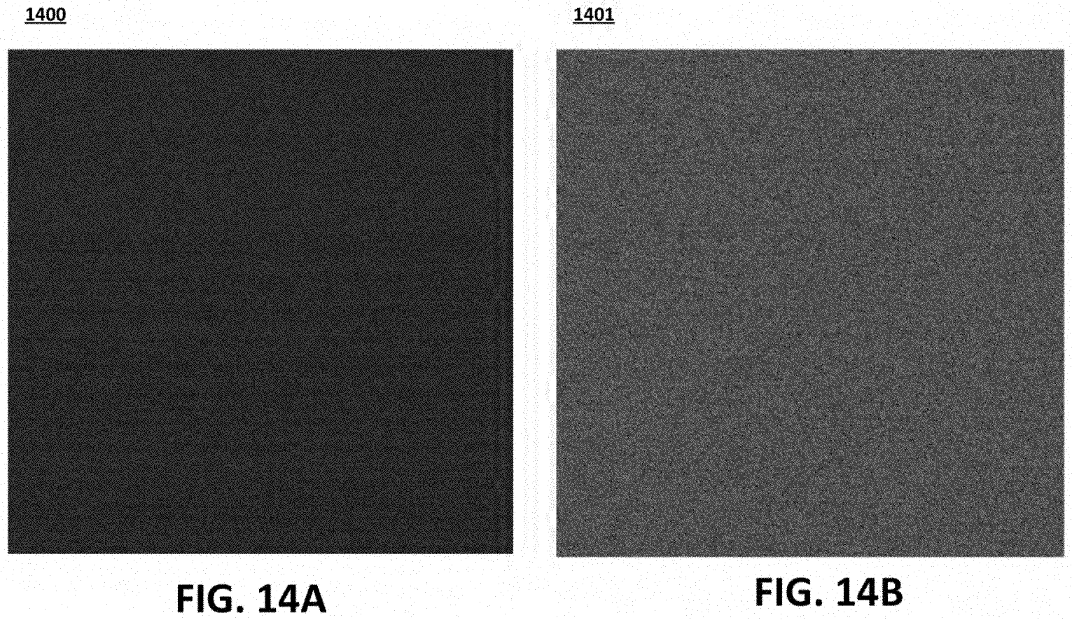

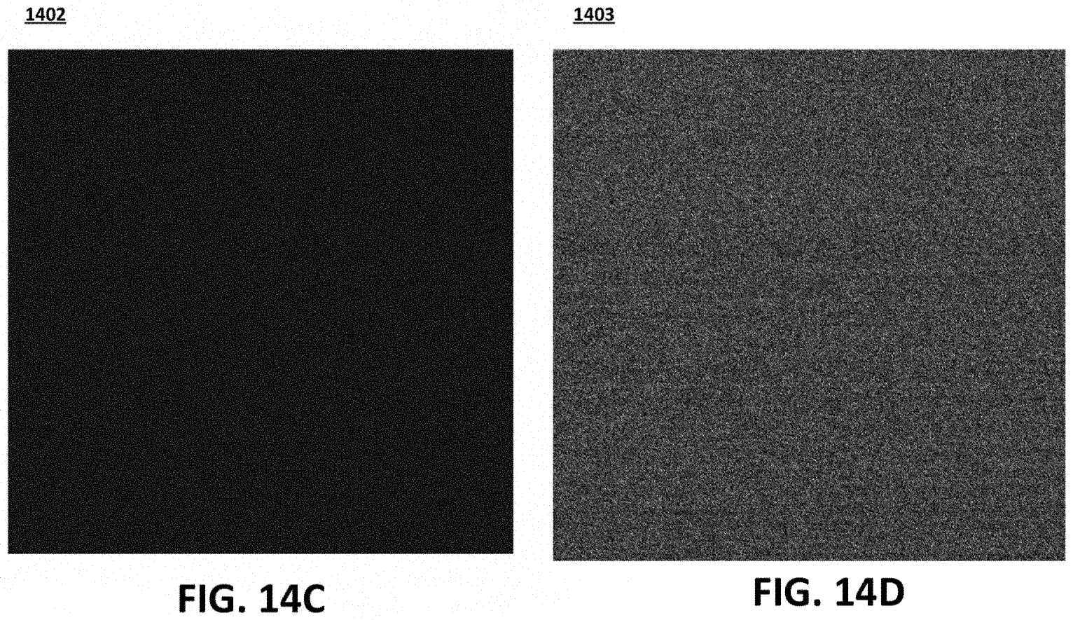

An operational example of one or more first allele or second allele image representations 1400-1403 that may be generated is depicted in FIGS. 14A-D. In this particular example, a DNA nucleotide input feature type designation is portrayed such that an image representation for each category associated with the DNA nucleotide input feature type designation is generated. In this case, each image representation corresponding to a category of the DNA nucleotide input feature type designation also corresponds to a unique color when indicating the presence of the corresponding feature value in the input feature for a particular image representation region. However, it will also be appreciated by one of skill in the art that each image representation from each category may be combined into a single image representation where each color uniquely represents a DNA nucleotide input feature type designation category. For example, a DNA nucleotide input feature type designation category of ‘A’ may correspond to a red color while a DNA nucleotide input feature type designation category of ‘C’ may correspond to a green color.

Once the first allele image representation and the second allele image representation are generated, one or more mathematical and/or logical operators may be applied to generate a differential image representation. A match and/or difference in the image region for the differential image representation may be indicated in a variety of ways, including using numerical values, colors, and/or the like. For example, a match between image regions in the first image representation and the second image representation may be indicated by an image region value of ‘1’ and a non-match between image regions in the first image representation and the second image representation may be indicated by an image region value of ‘0’. As another example, a match between image regions in the first image representation and the second image representation may be indicated by a white color in the corresponding image region while a non-match between image regions in the first image representation and the second image representation may be indicated by a black color in the corresponding image region.

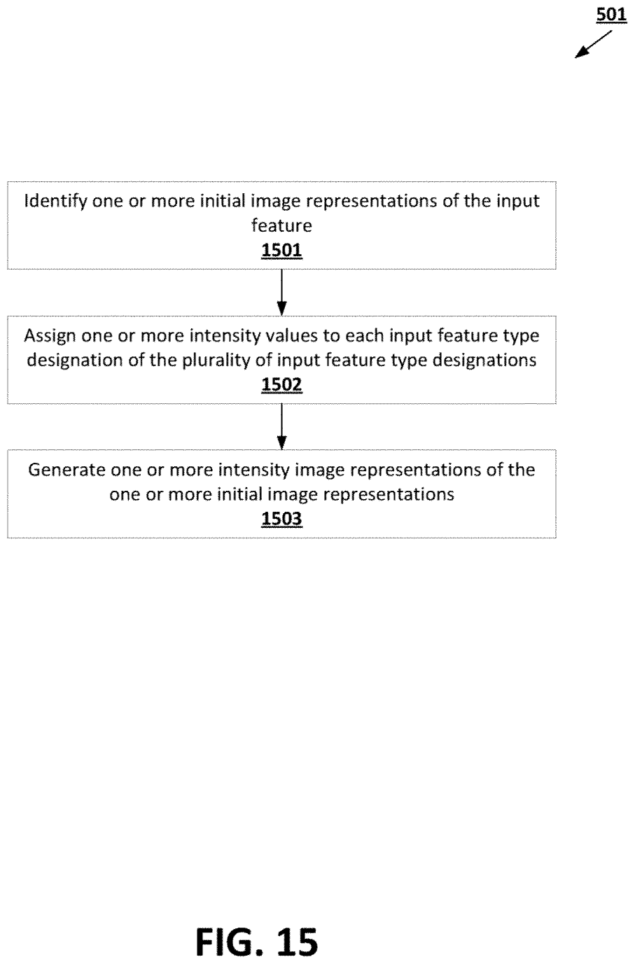

In some embodiments, step/operation 501 may be performed in accordance with the various steps/operations of the process that is depicted in FIG. 15, which is a flowchart diagram of an example process for generating an intensity image representation. The process that is depicted in FIG. 15 begins at step/operation 1501, when the predictive data analysis computing entity 106 identifies one or more initial image representations of the input feature. The one or more initial image representations may be generated by the process described in step/operation 402.

At step/operation 1502, the predictive data analysis computing entity 106 may assign one or more intensity values to each input feature type designation of the plurality of input feature type designations. In some embodiments, input feature type designations associated with feature values corresponding to a categorical feature type may have an intensity value assigned for each category of the input feature type designation. For example, a DNA nucleotide input feature type designation may be associated with categories ‘A’, ‘C’, ‘T’, ‘G’, and missing (corresponding to adenine, cytosine, thymine, and guanine, respectively) may be assigned intensity values 1, 0.75, 0.5, 0.25, and 0. Additionally or alternatively, the categories ‘A’, ‘C’, ‘T’, ‘G’, and missing may be assigned intensity values corresponding to the colors red, green, blue, white, and black, respectively. In some embodiments, input feature type designations associated with feature values corresponding to a numeric feature type may have an intensity value based at least in part on the numeric value of the feature value. For example, a MAF input feature type designation may be associated with a numeric value between 0 and 1. As such, a feature value of ‘0.3’ for a MAF input feature type designation may be associated with an intensity value of 0.3. In some embodiments, intensity value for a feature value corresponding to a numeric input feature type may be rounded to the nearest integer or decimal place of interest. For example, a feature value of 0.312 for a MAF input feature type designation may be associated with an intensity value of 0.3.

At step/operation 1503, the predictive data analysis computing entity 106 may generate one or more intensity image representations of the one or more initial image representations. In some embodiments, the predictive data analysis computing entity 106 may generate the one or more intensity image representation based at least in part on the one or more feature values and the assigned intensity value for each input feature type designation.

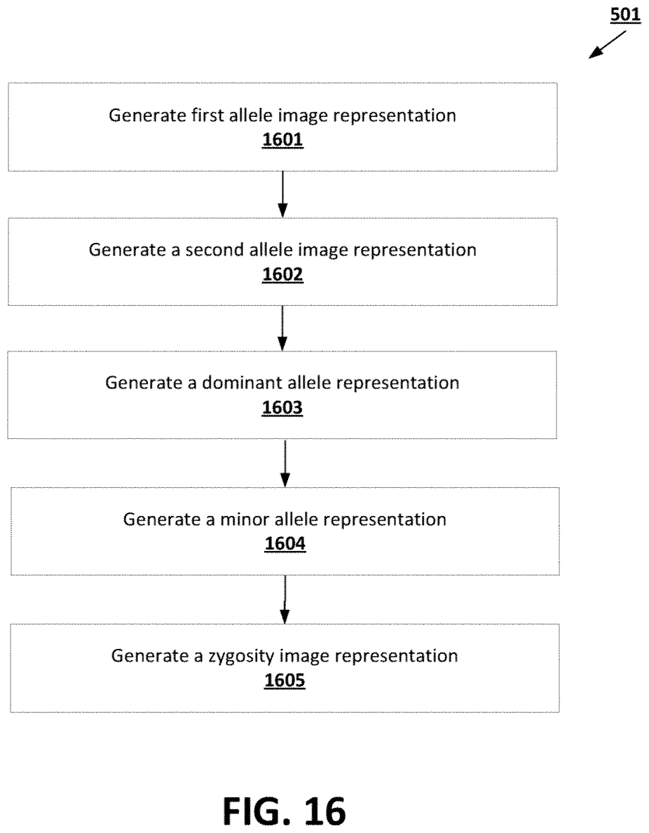

In some embodiments, step/operation 501 may be performed in accordance with the various steps/operations of the process that is depicted in FIG. 16, which is a flowchart diagram of an example process for generating a zygosity image representation. The process that is depicted in FIG. 16 begins at step/operation 1601, when the predictive data analysis computing entity 106 generates a first allele image representation. In some embodiments, an input feature may describe a representation of a genetic sequence associated with an individual, as indicated by feature values of an input feature associated with an individual. In some embodiments, the genetic sequence corresponds to one or more particular genes and/or alleles for a first chromosome and/or a first set of chromosomes of the individual. The first allele image representation may be generated substantially similarly to the process described in step/operation 402.

At step/operation 1602, the predictive data analysis computing entity 106 generates a second allele image representation. In some embodiments, an input feature may describe a representation of a genetic sequence associated with an individual, as indicated by feature values of an input feature associated with an individual. In some embodiments, the genetic sequence corresponds to one or more particular genes and/or alleles for a second chromosome and/or a second set of chromosomes of the individual. In some embodiments, the individual associated with the second allele image is the same individual associated with the first allele image representation. In some embodiments, the individual associated with the second allele image is a different individual than the individual associated with the first allele image representation. The second allele image representation may be generated substantially similarly to the process described in step/operation 402.

At step/operation 1603, the predictive data analysis computing entity 106 generates a dominant allele image representation. In some embodiments, an input feature may describe a representation of a genetic sequence associated with a dominant genetic sequence for a particular genetic sequence, as indicated by feature values of an input feature. In some embodiments, the genetic sequence corresponds to a particular gene and/or allele. In some embodiments, the dominant genetic sequence is the genetic sequence most common in a population. The dominant allele image representation may be generated substantially similarly to the process described in step/operation 402.

At step/operation 1604, the predictive data analysis computing entity 106 generates a minor allele image representation. In some embodiments, an input feature may describe a representation of a genetic sequence associated with a minor genetic sequence for a particular genetic sequence, as indicated by feature values of an input feature. In some embodiments, the genetic sequence corresponds to a particular gene and/or allele. In some embodiments, the minor genetic sequence is the genetic sequence associated with a second most common genetic sequence in a population. In some embodiments, the minor genetic sequence is a genetic sequence associated other than the most common genetic sequence in a population. The minor allele image representation may be generated substantially similarly to the process described in step/operation 402.

At step/operation 1605, the predictive data analysis computing entity 106 generates a zygosity image representation. In some embodiments, a representation of a zygosity associated with an individual based at least in part on an associated first allele image representation and a second allele image representation for the individual, a dominant allele representation, and a minor allele representation for a genetic sequence (e.g. gene, allele, chromosome, etc.). In some embodiments, the zygosity image representation may be generated based at least in part on a comparison between the first allele image representation and a second allele image representation using one or more mathematical and/or logical operators, similar to the differential image representation. Further, the zygosity image representation may be generated based at least in part on a comparison between the first allele image representation, the second allele image representation, the dominant allele representation, and the minor allele representation using one or more mathematical and/or logical operators. For example, if an individual is associated with a first allele image representation indicating a feature value of ‘A’ in the image region corresponding to the second genetic variant identifier and a second allele image representation indicates a feature value of ‘C’ in the image region corresponding to the second genetic variant identifier, the feature value for the second genetic variant identifier is determined to be heterozygous.

As another example, if an individual is associated with a first allele image representation indicating a feature value of ‘A’ in the image region corresponding to the first genetic variant identifier and a second allele image representation indicates a feature value of ‘A’ in the image region corresponding to the first genetic variant identifier, the feature value for the first genetic variant is determined to be homozygous. Further, the homozygous feature value of ‘A’ may be compared to the feature values corresponding to the first genetic variant identifier in the dominant allele image representation and/or the minor allele image representation. If the homozygous feature value matches the feature value in the dominant allele image representation, the feature value is determined to be homozygous with a dominant allele. If the homozygous feature value matches the feature value in the minor allele image representation, the feature value is determined to be homozygous with a minor allele. A heterozygous, homozygous with a dominant allele, homozygous with a minor allele, etc. may be indicated in a variety of ways, including using values corresponding to each category, colors corresponding to each category, etc. For example, an image region determined to be heterozygous may be associated with a value of ‘0’, an image region determined to be homozygous with a dominant allele may be associated with a value of ‘1’, and an image region determined to be homozygous with a dominant allele may be associated with a value of ‘2’.

As another example, an image region determined to be heterozygous may be associated with a green color, an image region determined to be homozygous with a dominant allele may be associated with a red color, and an image region determined to be homozygous with a dominant allele may be associated with a blue color.

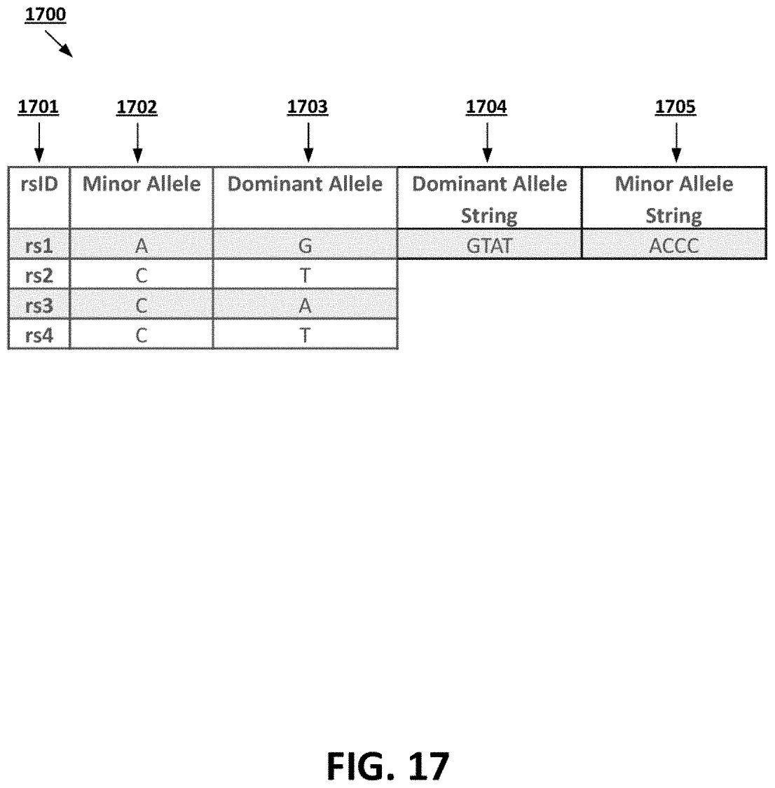

An operational example of an input feature 1700 that may be used to generate a zygosity image representation is depicted in FIG. 17. The input feature 1700 may comprise one or more feature values for both the minor allele 1702 and the dominant allele 1703 corresponding to one or more genetic variants 1701. Based at least in part on these one or more feature values provided by the input feature, a dominant allele value 1704 and a minor allele value 1705 may be determined.

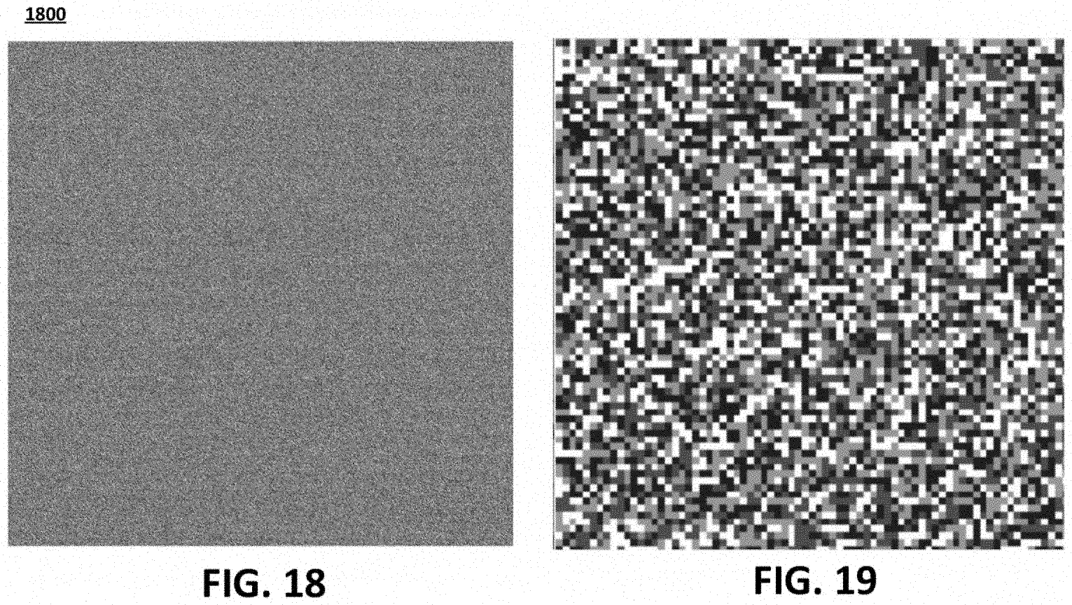

An operational example of a first allele image representation, second allele image representation, dominant allele image representation, or minor allele image representation 1800 that may be used in part to generate a zygosity image representation is depicted in FIG. 18. By way of example, a DNA nucleotide input feature type designation is portrayed. In this case, the image representation corresponding to a category of the DNA nucleotide input feature type designation also corresponds to a unique color when indicating the presence of the corresponding feature value in the input feature for a particular image representation region. For example, a DNA nucleotide input feature type designation category of ‘A’ may correspond to a red color, a DNA nucleotide input feature type designation category of ‘C’ may correspond to a green color, a DNA nucleotide input feature type designation category of ‘G’ may correspond to a blue color, a DNA nucleotide input feature type designation category of ‘T’ may correspond to a white color, and a DNA nucleotide input feature type designation category of ‘missing’ may correspond to a black color.

A zoomed in version of the operational example depicted in FIG. 18 is depicted in FIG. 19. In FIG. 19, the individual colors each corresponding to an image representation region, which further corresponds to a genetic variant identifier, is shown more clearly.

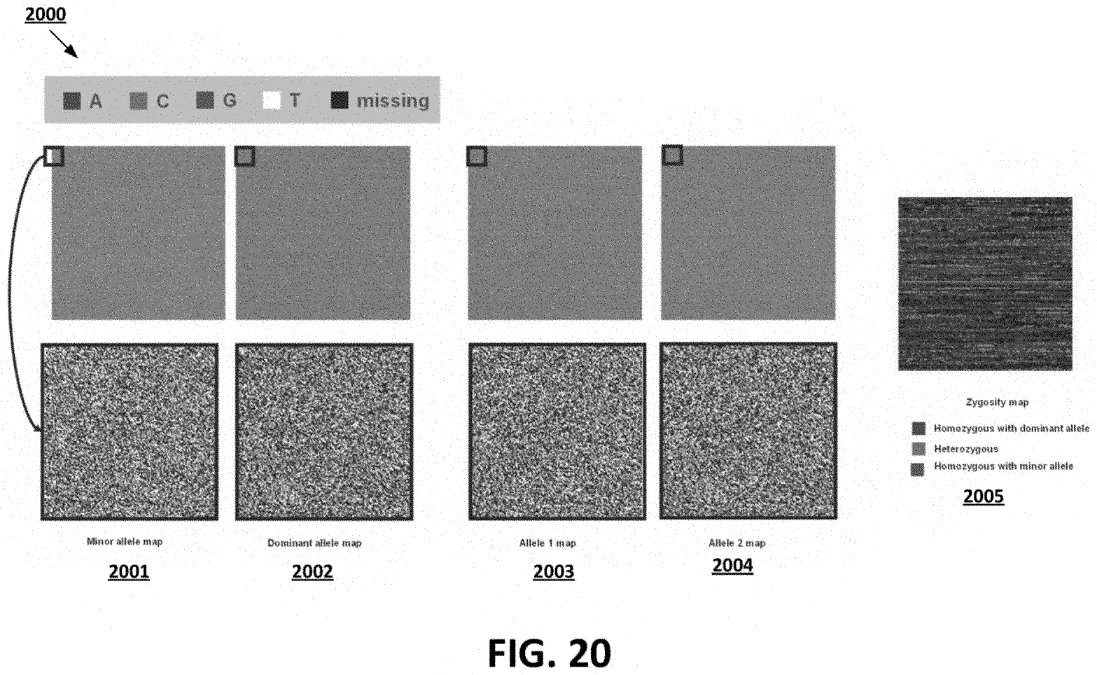

An operational example of a minor allele image representation 2001, a dominant image representation 2002, a first allele image representation 2003, a second allele image representation 2004, and a zygosity image representation 2005 is depicted in FIG. 20. The predictive data analysis computing entity 106 may generate the zygosity image representation 2005 based at least in part on an associated first allele image representation and a second allele image representation for the individual, a dominant allele representation, and a minor allele representation for a genetic sequence (e.g. gene, allele, chromosome, etc.). In some embodiments, the zygosity image representation may be generated based at least in part on a comparison between the first allele image representation and a second allele image representation using one or more mathematical and/or logical operators, similar to the differential image representation. Further, the zygosity image representation may be generated based at least in part on a comparison between the first allele image representation, the second allele image representation, the dominant allele representation, and the minor allele representation using one or more mathematical and/or logical operators.

Returning to FIG. 5, at step/operation 502, the predictive data analysis computing entity 106 generates a tensor representation of the one or more image representations. In some embodiments, to generate the tensor representation, the predictive data analysis computing entity 106 retrieves configuration data for a particular image-based processing routine from the model definition data 121 stored in the storage subsystem 108. However, one of ordinary skill in the art will recognize that the predictive data analysis computing entity 106 may generate the one or more images by applying any suitable technique for transforming the input feature into the one or more images. In some embodiments, the predictive data analysis computing entity 106 selects a suitable image-based processing routine for the tensor representation given the one or more properties of the input feature (e.g., inclusion of input feature type designations for the input feature, an indication of feature values pertaining to one or more individuals, and/or the like). In some embodiments, the predictive data analysis computing entity 106 may select a suitable image-based processing routine for the input feature based at least in part on a user specified preference. In some embodiments, the user specified preference may be indicated in the input feature.

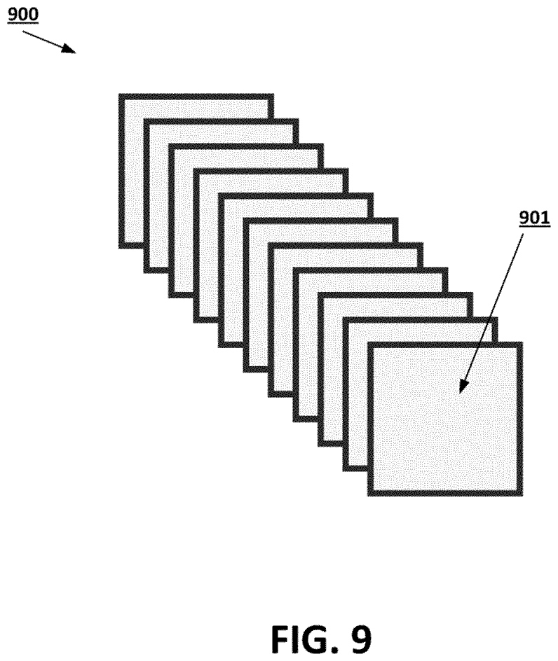

An operational example of generating a tensor representation 900 of the one or more image representations is depicted in FIG. 9. Each image representation 901 in the tensor representation 900 corresponds to an image representation generated by the predictive data analysis computing entity 106. By way of continuing example, the tensor representation 900 may comprise 4 image representations corresponding to the DNA Nucleotide input feature type designation and 1 image representation corresponding to the MAF input feature type designation.

At step/operation 503, the predictive data analysis computing entity 106 generates a plurality of positional encoding maps. In some embodiments, to generate the positional encoding maps, the predictive data analysis computing entity 106 retrieves configuration data for a particular image-based processing routine from the model definition data 121 stored in the storage subsystem 108. However, one of ordinary skill in the art will recognize that the predictive data analysis computing entity 106 may generate the plurality of positional encoding maps by applying any suitable technique for generating a plurality of positional encoding maps. In some embodiments, the predictive data analysis computing entity 106 selects a suitable image-based processing routine for the plurality of positional encoding maps given the one or more properties of the input feature (e.g., inclusion of input feature type designations for the input feature, an indication of feature values pertaining to one or more individuals, and/or the like). In some embodiments, the predictive data analysis computing entity 106 may select a suitable image-based processing routine for the plurality of positional encoding maps based at least in part on a user specified preference. In some embodiments, once the plurality of positional encoding maps are generated, they may be incorporated into the tensor representation

A positional encoding map may be comprised of positional encoding map regions each corresponding to a genetic variant identifier. Each region of a positional encoding map may correspond to an identifier value. For example, the first positional encoding map region may comprise an identifier value of ‘1’, the second positional encoding map region may comprise an identifier value of ‘2’, etc. In some embodiments, a positional encoding map set may comprise each positional encoding map region corresponding to the same genetic variant identifier across the plurality of positional encoding maps. For example, if the plurality of positional encoding maps comprise two positional encoding maps, and the positional encoding map regions corresponding to the first genetic variant identifier in both positional encoding maps comprise an identifier value of ‘1’, the positional encoding map region set for the first genetic variant identifier may comprise the identifier values ‘1,1’. In some embodiments, the identifier values of the positional encoding map corresponding to each positional encoding map regions are the same. In some embodiments, the identifier values of the positional encoding map corresponding to each positional encoding map regions are different.

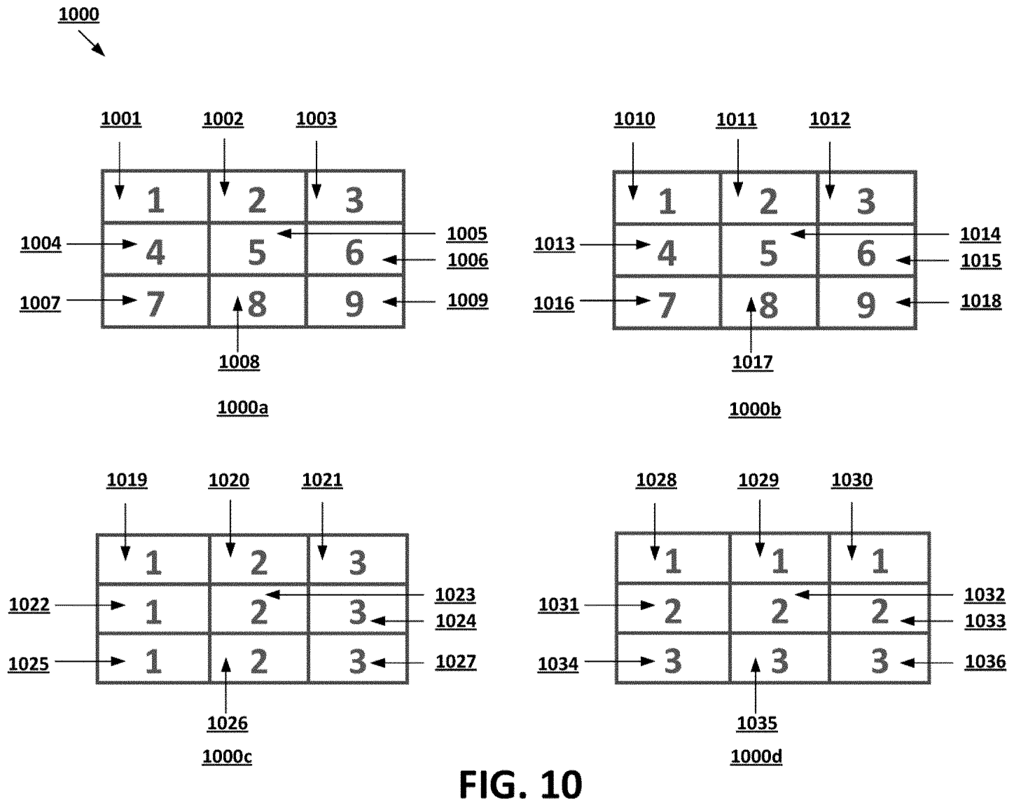

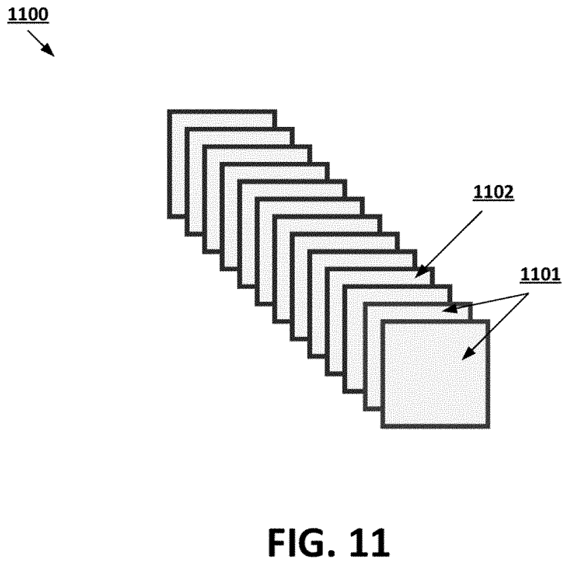

An operational example of a set of positional encoding maps 1000 is depicted in FIG. 10. In this particular example, the set of positional encoding maps 1000 comprises two positional encoding maps 1000a and 1000b. Each positional encoding map comprises a plurality of positional encoding map regions 1001-1009 for positional encoding map 1000a and positional encoding map regions 1010-1018 for positional encoding map 1000b. Each positional encoding map region corresponds to a genetic variant identifier. In some embodiments, the number of positional encoding map regions is based at least in part on the image representation configuration, as described with reference to FIG. 6. The value for each positional encoding map region may be assigned an identifier value. An identifier value may be any value, such as a numeric value, color, symbols, etc. For example, positional encoding map 1000a has 9 positional encoding map regions comprising the values 1-9, respectively. Similarly, positional encoding map 1000b has 9 positional encoding map regions comprising the values 1-9, respectively.

In some embodiments, one or more positional encoding map regions may comprise the same value. For example, positional encoding map 1000c includes positional encoding map regions 1019, 1022, and 1025, which are assigned the same identifier value. Similarly, positional encoding map 1000d includes positional encoding map regions 1028, 1029, and 1030, which are assigned the same identifier value.

A positional encoding map region set is comprised of each positional encoding map region from amongst the plurality of positional encoding maps corresponding to the same genetic variant identifier. For example, a positional encoding map region set for the genetic variant identifier rs1 may comprise the positional encoding map regions 1001 and 1010 from positional encoding map 1000a and 1000b, respectively. As such, the positional encoding map region set may correspond to ‘1,1’. As such, the genetic variant identifier rs1 may be assigned the positional encoding map region set corresponding to ‘1,1’ such that no other genetic variant identifier is assigned the positional encoding map region set. As another example, the positional encoding map region set for the genetic variant identifier rs2 may comprise the positional encoding map regions 1002 and 1012 from positional encoding map 1000a and 1000b, respectively. As such, the positional encoding map region set may correspond to ‘2,2’. As such, the genetic variant identifier rs2 may be assigned the positional encoding map region set corresponding to ‘2,2’. As another example, a positional encoding map region set for the genetic variant identifier rs1 may comprise the positional encoding map regions 1019 and 1028 from positional encoding map 1000c and 1000d, respectively. As such, the positional encoding map region set may correspond to ‘1,1’. As such, the genetic variant identifier rs1 may be assigned the positional encoding map region set corresponding to ‘1,1’ such that no other genetic variant identifier is assigned the positional encoding map region set. As another example, the positional encoding map region set for the genetic variant identifier rs2 may comprise the positional encoding map regions 1020 and 1029 from positional encoding map 1000c and 1000d, respectively. Accordingly, the positional encoding map region set may correspond to ‘2,1’. As such, the genetic variant identifier rs2 may be assigned the positional encoding map region set corresponding to ‘2,1’.

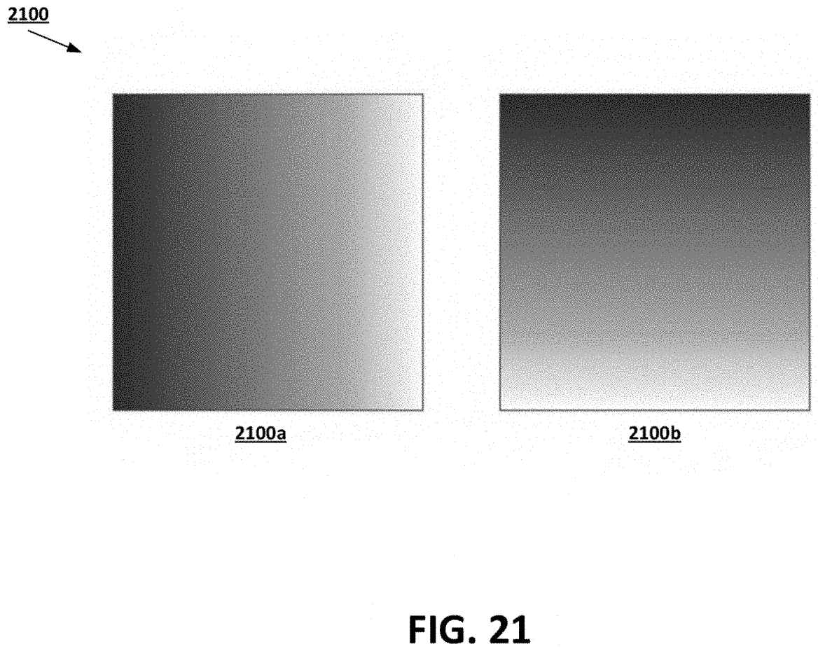

Another operational example of a set of positional encoding maps 2100 is also depicted in FIG. 21. In this particular example, the set of positional encoding maps 2100 comprises two positional encoding maps 2100a and 2100b. The positional encoding map region set is comprised of a unique set of intensity values from which a genetic variant identifier may be identified.

Returning to FIG. 5, at step/operation 504, the predictive data analysis computing entity 106 generates the initial input feature representation by incorporating the set of positional encoding maps into the tensor representation. In some embodiments, the predictive data analysis computing entity 106 appends the set of positional encoding maps to the image representations of the tensor representation to generate the initial input feature representation.

An operational example of incorporating a set of positional encoding maps into the tensor representation 1100 is depicted in FIG. 11. The tensor representation comprising the one or more generated image representations 1102 may additionally incorporate the set of positional encoding maps 1101. In some embodiments, the set of positional encoding maps may uniquely identify a particular genetic variant identifier present in the one or more image representations 1102.

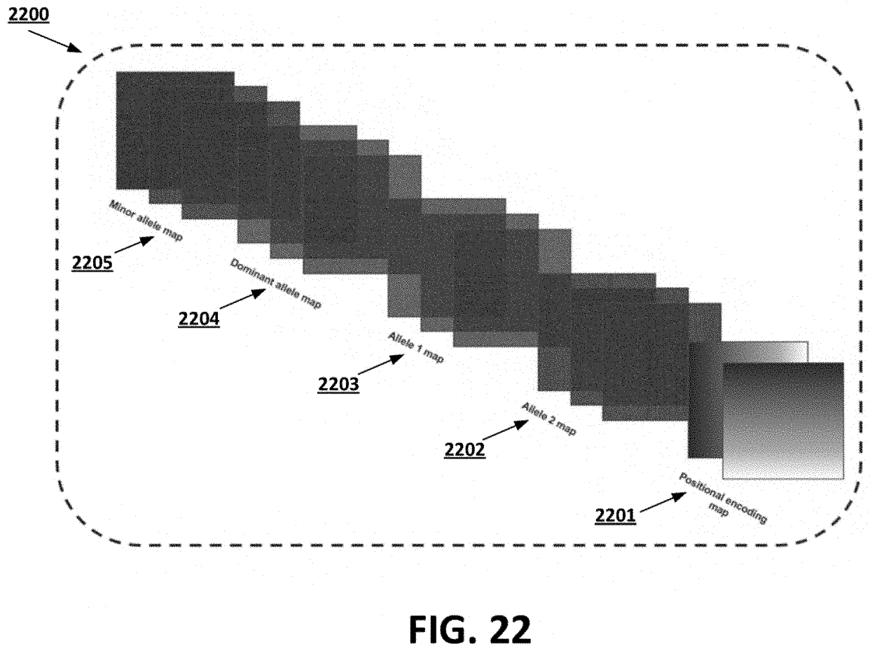

Another operational example of incorporating the plurality of positional encoding maps into the tensor representation 2200 is depicted in FIG. 22. The tensor representation comprising the one or more generated image representations 2202-2205 may additionally incorporate the plurality of positional encoding maps 2201. In some embodiments, the plurality of positional encoding maps may uniquely identify a particular genetic variant identifier present in the one or more image representations 1102. In this example, the tensor representation includes one or more image representations for a second allele image representation 2202, one or more image representations for a first allele image representation 2203, one or more image representations for a dominant allele image representation 2204, one or more image representations for a minor allele image representation 2205, and a plurality of positional encoding maps 2201.

B. Generating Multi-Segment Input Feature Representations

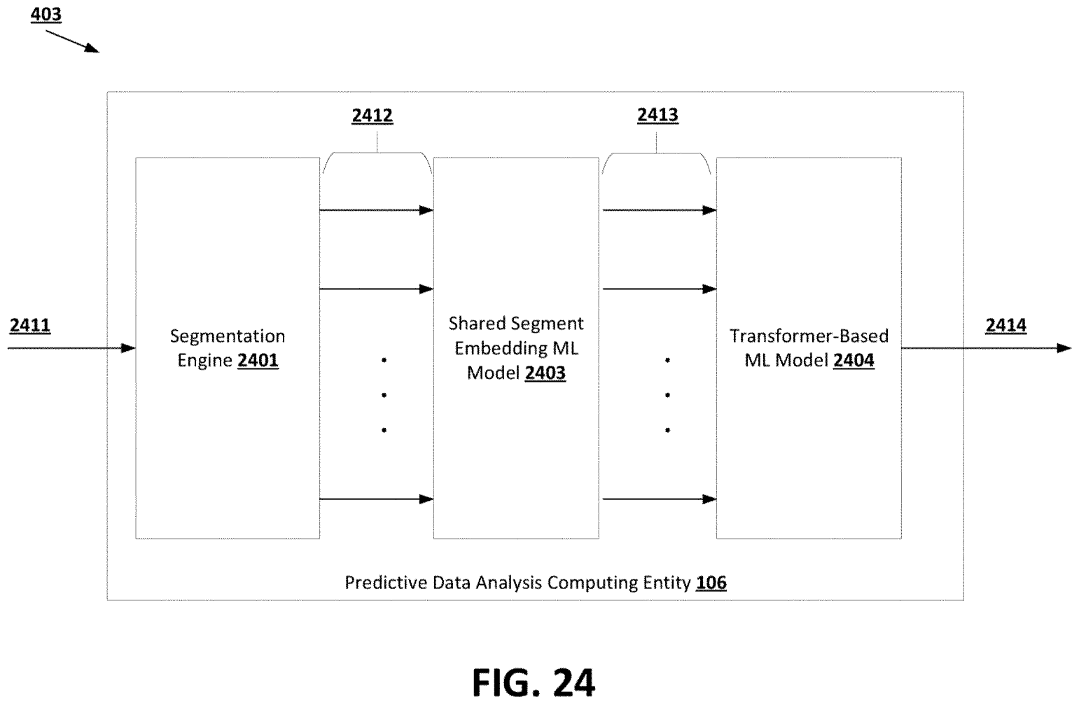

In some embodiments, step/operation 403 may be performed in accordance with the process that is depicted FIG. 24. The process that is depicted in FIG. 24 begins when a segmentation engine 2401 generates m input feature representation segments 2412 of the initial input representation 2411, where each input representation segment belongs to an input feature representation super-segment of c input feature representation segments.

In some embodiments, the initial input feature representation segment comprises an ordered sequence of n input feature representation values. The ordered sequence is in turn associated with g genetic variant identifiers and c chromosome designations, such that each genetic variant is associated with a corresponding variant-related subsequence of the ordered sequence and each chromosome designation is associated with a chromosome-related subsequence of the ordered sequence. In other words, in some embodiments, the ordered sequence comprises disjoint c chromosome-related subsequences, each including those input feature representation values that are associated with genetic variant identifiers (e.g., SNPs) of a particular chromosome, and the ordered sequence comprises g disjoint variant-related subsequences each including those input feature representation values that are associated with a particular genetic variant identifier (e.g., a particular SNP). In this way, each chromosome-related subsequence that is associated with a particular chromosome designation comprises all of the variant-related subsequences for those genetic variant identifiers (e.g., those SNPs) that are associated with the particular chromosome designation.

In some embodiments, the ordered sequence of n input feature representation values may be divided into c input feature representation super-segments, where each input feature representation segment is associated with a corresponding chromosome designation and comprises the chromosome-related subsequence for the corresponding chromosome designation. Accordingly, the ordered sequence of n input feature representation values can be divided into disjoint segments that are determined based at least in part on disjoint chromosome-related subsequences associated with the c chromosome designations. For example, where c=46, the ordered sequence of n input feature representation values may be divided into 46 input feature representation super-segments, where each input feature representation super-segment includes those input feature representation values (e.g., those genetic variant identifier values) that correspond to a particular chromosome of 46 chromosomes. Accordingly, chromosome-based demarcations can be used to create one level of segmentation across the ordered sequence of n input feature representation values. As described below, the first-level segments can then in turn be further segmented in accordance with a segmentation policy to generate second-level segments, referred to herein as input feature representation segments.

In some embodiments, given c input feature representation super-segments, an ith input feature representation super-segment that is associated with an ith chromosome designation of c chromosome designations can be divided into mᵢ input feature representation segments. Accordingly, each input feature representation super-segment may be further segmented to create a set of input feature representation segments that are determined based at least in part on the input feature representation super-segment, and then each generated set of input feature representation segments may be combined across all of the c input feature representation super-segments to generate m input feature representation segments. For example, given c=46, each of the 46 resulting input feature representation super-segments may be divided into a resulting set of mᵢ input feature representation segments, and then the 46 resulting sets may be combined across the 46 input feature representation super-segments to generate a set of Σᵢ₌₁⁴⁶mᵢ=m input feature representation segments.

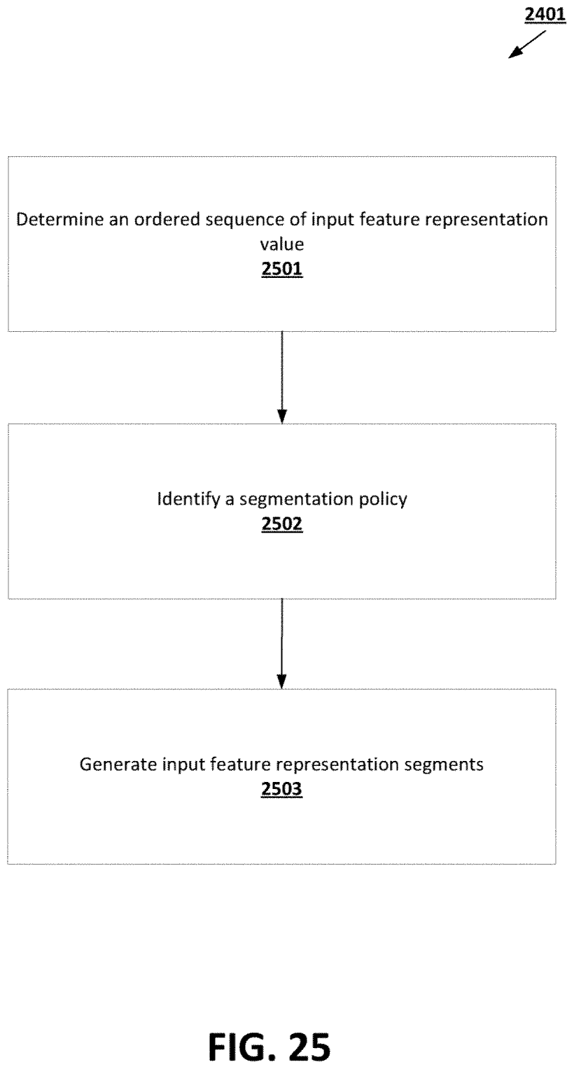

In some embodiments, the segmentation engine 2401 is configured to perform the steps/operations of the process that is depicted in FIG. 25. The process that is depicted in FIG. 25 begins at step/operation 2501 when the predictive data analysis computing entity 106 determines an ordered sequence of n input feature representation values of an initial input feature representation. In some embodiments, if the initial input feature representation is a one-dimensional vector of n values, then an ordered sequence of n input feature representation values may be generated for the initial input feature representation by ordering the n values in accordance with the order defined by the respective positions of the vector. In some embodiments, if the initial input feature representation is a two-dimensional matrix of \sqrt{n}*\sqrt{n} values, then the ordered sequence of n input feature representation values may be generated by defining an ordering of rows and columns of the two-dimensional matrix, such that a matrix value that belongs to an ath row of A rows of the matrix and a bth column of B columns of the matrix may either be associated with an (a+A*b)th in-sequence position indicator in an ordered sequence of n input feature representation values or an (a*B+b)th in-sequence position indicator in an ordered sequence of n input feature representation values. Similar logics may be applied to generate an ordered sequence values for initial input feature representations having three or more dimensions.

For example, if the initial input feature representation is a one-dimensional vector of n values, then an ordered sequence of n input feature representation values may be generated for the initial input feature representation by ordering the n values in accordance with the order defined by the respective positions of the vector. Afterward, based at least in part on the ordered sequence, each of the n input feature representation values may be associated with an in-sequence position indicator that describes where in the ordered sequence the input feature representation value is (for example, a first value in the ordered sequence may be associated with an in-sequence position indicator of one, a second value in the ordered sequence may be associated with an in-sequence position indicator of two, and so on). Thereafter, each input feature representation segment may be generated as a subset of the ordered sequence that comprises all those input feature representation values starting with an ath input feature representation value in the ordered sequence and ending with a bth input feature representation value in the ordered sequence, where a is the initial in-sequence position indicator for the noted input feature representation segment, and b is the terminal in-sequence position indicator for the noted input feature representation segment. In some embodiments, the segmentation policy, for each input feature representation segment, the initial in-sequence position indicator for the noted input feature representation segment and the terminal in-sequence position indicator for the noted input feature representation segment.

In some embodiments, the ordered sequence of the n input feature representation values is determined by: (i) determining an ordered sequence of c chromosome designations that assigns an in-sequence position indicator for each chromosome designation, (ii) for chromosome designation, determining an ordered input feature representation super-segment by ordering the input feature representation values that fall within the input feature representation super-segment for the chromosome designation, (iii) appending the ordered input feature representation super-segment in accordance with the ordered sequence of c chromosome designations such that an ith ordered input feature representation super-segment for an ith chromosome designation comes before the (i+1th) ordered input feature representation super-segment for the (i+1)th chromosome designation if the (i+1th) ordered input feature representation super-segment exists, and (iv) determining the ordered sequence based at least in part on the output of the appending performed in (iii).

At step/operation 2502, the predictive data analysis computing entity 106 identifies a segmentation policy. The segmentation policy may define: (i) for each chromosome designation of c chromosome designations associated with an input feature, an intra-chromosome segment count (i.e., an mᵢ value as described above), and (ii) a shared per-segment input feature representation value count that is common across m input feature representation segments generated based at least in part on the segmentation policy (where m=Σmᵢ, with i iterating over the c chromosome designations). An intra-chromosome segment count for a particular chromosome designation may describe a recommended number of input representation segments that should be generated based at least in part on the input feature representation super-segment for the chromosome designation. For example, if a particular chromosome designation is associated with an intra-chromosome segment count of 20, then the input feature representation super-segment for the particular chromosome designation should be segmentized to generate 20 input feature representation segments. In an exemplary embodiment, if the described particular chromosome designation is one of 2 total chromosome designations, with the other chromosome designation being associated with an intra-chromosome segment count of 30, then a total of 20+30 input feature representation segments may be generated based at least in part on the described segmentation policy.

The shared per-segment input feature representation value count may describe the required/recommended number of input feature representation values from an ordered sequence of input feature representation values that should be in each input feature representation segment. For example, the shared per-segment input feature representation value count may require that each input feature representation value should include 10 input feature representation values. In some embodiments, given a segmentation policy that defines a particular intra-chromosome segment count mᵢ for an input feature representation super-segment ssᵢ, as well as a particular shared per-segment input feature representation value count v, then the input feature representation values that fall within ssᵢ should be divided into mᵢ subsets (e.g., mᵢ disjoint subsets, mᵢ overlapping subsets, and/or the like), where each of the mᵢ subsets includes v of the input feature representation values that fall within ssᵢ. This may in an exemplary embodiment include, given mᵢ=2, v=20, and a total of 30 input feature representation values that fall within ssᵢ, generating a first input feature representation segment that starts with a first input feature representation value of the 30 input feature representation values that fall within ssᵢ and ends with a twentieth input feature representation value of the 30 input feature representation values that fall within ssᵢ, as well as a second input feature representation segment that starts with an eleventh input feature representation value of the 30 input feature representation values that fall within ssᵢ and ends with a thirtieth input feature representation value of the 30 input feature representation values that fall within ssᵢ.

In some embodiments, the segmentation policy defines: (i) the value of m (i.e., the number of input feature representation segments that should be determined based at least in part on the initial input feature representation for the input feature, which may in some embodiments be determined based at least in part on a value of the count of genetic variants associated with the input feature), and (ii) for each input feature representation segment of m defined input feature representation segments, an initial input feature representation value, a terminal input feature representation value, and a segment length indicator. In some embodiments, the segment length indicator is a value that describes a deviation between the initial input feature representation value for a corresponding input feature representation segment and the terminal input feature representation value for the corresponding input feature representation segment. For example, if an input feature representation segment is defined to include all input feature representation values beginning with a 100^{th} input feature representation value in an ordered sequence and ending with a 200^{th} input feature representation value in the ordered sequence, then the input feature representation segment may be associated with a segment length indicator of 100 that describes that the input feature representation segment is associated with 100 input feature representation values in the ordered sequence.

In some embodiments, the segmentation policy defines, for each input feature representation segment, the initial in-sequence position indicator for the noted input feature representation segment and the terminal in-sequence position indicator for the noted input feature representation segment. In some embodiments, the m input feature representation segments generated based at least in part on the segmentation policy are associated with a segment order that describes an ordered segment sequence of the m input feature representation segments, where the segment order defines a segment in-sequence positional indicator for each input feature representation segment that describes where in the ordered sequence of the m input feature representation segments the input feature representation segment is.

In some embodiments, the ordered segment sequence of the m input feature representation segments is determined by ordering the m input feature representation segments based at least in part on the initial in-sequence position indicators for the m input feature representation segments, such that an ith input feature representation segment in the ordered sequence has an initial in-sequence position indicator that is smaller than the initial in-sequence position indicator for a jth input feature representation segment in the ordered sequence, where i<j. In some embodiments, the ordered sequence of the m input feature representation segments is determined by ordering the m input feature representation segments based at least in part on the terminal in-sequence position indicators for the m input feature representation segments, such that an ith input feature representation segment in the ordered sequence has a terminal in-sequence position indicator that is smaller than the terminal in-sequence position indicator for a jth input feature representation segment in the ordered sequence, where i<j.

At step/operation 2503, the predictive data analysis computing entity 106 generates the m input feature representation segments by applying the segmentation policy to the ordered sequence of the n input feature representation values. As described above, in some embodiments, the segmentation policy may define where each input feature representation segment should begin and end in the ordered sequence. Therefore, by applying the segmentation policy to the ordered sequence of the n input feature representation values, the predictive data analysis computing entity 106 may be able to generate the m input feature representation segments with O(m) computational complexity.

In some embodiments, the m input feature representation segments comprise, for each chromosome designation, a chromosome-related segment subset of the m input feature representation segments that comprises those input feature representation segments that are generated by segmentizing the input feature representation super-segment for the chromosome designation. For example, for a first chromosome designation in an ordered sequence of c chromosomes, if m₁ for the first chromosome designation is 15, then the first chromosome designation may be associated with the first 15 input feature representation segments in an ordered sequence of m input feature representation segments. As such, the chromosome-related segment subset of the m input feature representation segments for the first chromosome designation may comprise the first 15 input feature representation segments in the ordered sequence of m input feature representation segments.

Returning to FIG. 24, after the m input feature representation segments 2412 are generated by the segmentation engine 2401, the m input feature representation segments 2412 are processed by a shared segment embedding machine learning model 2403 to generate m segment-wise representations 2413 that comprise a respective segment-wise representation for each of the m input feature representation segments 2412. In some embodiments, the shared segment embedding machine learning model 2403 is configured to, for each input feature representation segment of the m input feature representation segments 2412: (i) generate a fixed-size data representation, and (ii) process the fixed-size data representation for the input feature representation segment using one or more machine learning layers (e.g., one or more feedforward neural network layers) to generate the segment-wise representation for the input feature representation segment. In some embodiments, each segment-wise representation generated by the shared segment embedding machine learning model 2403 is a fixed-size segment embedding for the corresponding input feature representation segment.

After the m segment-wise representations 2413 are generated by the shared segment embedding machine learning model 2403, the m segment-wise representations 2413 are processed by a transformer-based machine learning model 2404 to generate the multi-segment input feature representation 2414. In some embodiments, the transformer-based machine learning model 2404 is a transformer-based machine learning model (e.g., a bidirectional transformer-based machine learning model, such as a Bidirectional Encoder Representations from Transformers (BERT) machine learning model) that is configured to process m segment-wise transformer input data objects comprising a respective segment-wise transformer input data object for each of the segment-wise transformer m input feature representation segments 2412 to generate the multi-segment input feature representation 2414, where the segment-wise transformer input data object for an input feature representation segment may be determined based at least in part on (e.g., may comprise) at least one of the following: (i) the segment-wise representation for the input feature representation segment, as generated by the shared segment embedding machine learning model 2403, (ii) the positional representation (e.g., a fixed-size positional embedding) of a segment in-sequence positional indicator for the input feature representation segment within an ordered segment sequence of the m input feature representation segments 2412, and (iii) a chromosome representation (e.g., a fixed-size chromosome embedding) of the corresponding chromosome designation associated with the input feature representation segment.

In some embodiments, the transformer-based machine learning model 2404 is a language-based machine learning model that processes segment-wise transformer input data objects based on sentence groupings of the underlying input feature representation segments. For example, the transformer-based machine learning model 2404 may treat each segment-wise transformer input data object as a word and each grouping of segment-wise transformer input data objects for input feature representation segments related to a particular chromosome designation as a sentence (e.g., a sentence that starts with a beginning-of-sentence token and ends with an end-of-sentence token).

In some embodiments, for an ith input feature representation segment within an ordered segment sequence of m input feature representation segments that is associated with a jth chromosome designation within an ordered chromosome sequence of c chromosome designations, the input feature data object for the noted input feature representation segment may comprise the segment-wise representation for the noted input feature representation segment, a positional representation that may be a fixed-size embedding of i (i.e., of the segment in-sequence positional indicator for the noted input feature representation segment), and a chromosome representation that may be a fixed size embedding of the jth chromosome (i.e., of the corresponding chromosome designation associated with the noted input feature representation segment). The m segment-wise transformer input data objects for the m input feature representation segments 2412 may then be processed by the transformer-based machine learning model 2404 to generate the multi-segment input feature representation 2414.

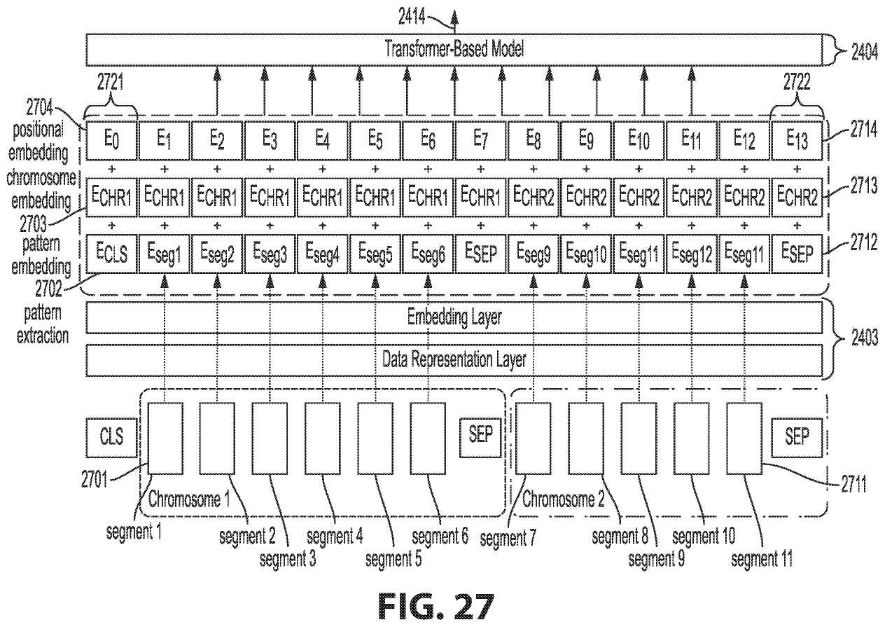

An operational example of generating a multi-segment input feature representation is depicted in FIG. 27. As depicted in FIG. 27, the input data comprises 11 input feature representation segments associated with 2 input feature representation super-segments, with the first input feature representation super-segment being associated with a Chromosome 1 and the first six input feature representation segments, and the second input feature representation super-segment being associated with a Chromosome 2 and the last six input feature representation segments.

As further depicted in FIG. 27, for each input feature representation of the 11 input feature representation segments, a segment-wise transformer input data object is generated based at least in part on: (i) a segment-wise representation of the input feature representation that is generated using the data representation layer and the embedding layer of the shared segment embedding machine learning model 2403, (ii) a chromosome representation, and (iii) a positional representation. For example, the first input feature representation 2701 of the 11 input feature representation segments is associated with a segment-wise transformer input data object 2721 that is generated based at least in part on a segment-wise representation 2702, a chromosome representation 2703 for Chromosome 1, and a positional representation 2704 for the first position in the ordered sequence of the 11 input feature representation segments. As another example, the eleventh input feature representation 2711 of the 11 input feature representation segments is associated with a segment-wise transformer input data object 2722 that is generated based at least in part on a segment-wise representation 2712, a chromosome representation 2713 for Chromosome 2, and a positional representation 2714 for the eleventh position in the ordered sequence of the 11 input feature representation segments. As further depicted in FIG. 27, the 11 segment-wise transformer input data objects for the 11 input feature representation segments are processed by the transformer-based machine learning model 2404 to generate the multi-segment input feature representation 2414.

Accordingly, as described below, various embodiments of the present invention address technical challenges related to efficiently performing machine learning tasks on large datasets and/or on data-intensive datasets. As described below, in various embodiments of the present invention, a large and/or data-intensive dataset is converted into input feature representation super-segments and input feature representation segments, where the input feature representation super-segments are mapped to sentences and input feature representation segments are mapped to words. Then, segment-wise representations for input feature representation segments are provided to a transformer-based language model in accordance with the sentence-word hierarchy described above to generate multi-segment input feature representations that can then be used to perform efficient and effective predictive data analysis operations. This highlights a major technical advantage of the noted embodiments of the present invention: instead of processing an initial input feature representation as a whole, the noted embodiments of the present invention first generate m input feature representation segments of the initial input feature representation, and then process the m input feature representation segments using efficient and effective transformer-based language models. As a result, instead of performing the often excessively large computational task of processing the initial input feature representation as a whole and using an excessively large amount of computational resources and a large amount of processing time, various embodiments of the present invention divide the noted computational task into smaller computational sub-tasks that can be more manageably executed using transformer-based language models and by utilizing the sentence-word hierarchy described above. In this way, various embodiments of the present invention enable faster and less-resource-intensive processing of large machine learning tasks and/or data-intensive machine learning tasks by hierarchically segmenting input spaces and using the noted hierarchical segmentations to enable transformer-based encoding of the noted input spaces.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for generating a multi-segment prediction based at least in part on an initial input feature representation, the computer-implemented method comprising:
determining, using one or more processors and based at least in part on the initial input feature representation, an ordered sequence of n input feature representation values, wherein: (i) the initial input feature representation is a fixed-size representation of an input feature comprising g feature values, (ii) each feature value corresponds to a genetic variant identifier of g genetic variant identifiers, (iii) each genetic variant identifier is associated with a chromosome designation of c chromosome designations and a corresponding variant-related subsequence of the ordered sequence, and (iv) each chromosome designation is associated with a chromosome-related subsequence of the ordered sequence;
generating, using the one or more processors and based at least in part on the ordered sequence, c input feature representation super-segments, wherein each input feature representation segment is associated with a corresponding chromosome designation and comprises the chromosome-related subsequence for the corresponding chromosome designation;
generating, using the one or more processors and based at least in part on the c input feature representation super-segments, m input feature representation segments of the ordered sequence, wherein the m input feature representation segments comprise, for each chromosome designation, a chromosome-related segment subset of the m input feature representation segments that comprises those input feature representation segments that are generated by segmentizing the input feature representation super-segment for the chromosome designation;
for each input feature representation segment, determining, using the one or more processors and a shared segment embedding machine learning model and based at least in part on the input feature representation segment, a segment-wise representation of the input feature representation segment;
determining, using the one or more processors and a transformer-based machine learning model and based at least in part on each segment-wise representation, a multi-segment input feature representation of the input feature;
generating, using the one or more processors and a downstream prediction machine learning model, and based at least in part on the multi-segment input feature representation, the multi-segment prediction; and
performing, using the one or more processors, one or more prediction-based actions based at least in part on the multi-segment prediction.

2. The computer-implemented method of claim 1, wherein determining the multi-segment input feature representation comprises:
determining an ordered segment sequence of the m input feature representation segments based at least in part on the ordered sequence;
for each input feature representation segment, determining a segment-wise transformer input data object based at least in part on the segment-wise representation of the input feature representation segment, a positional representation of a segment in-sequence positional indicator for the input feature representation segment within the ordered segment sequence, and a chromosome representation of the corresponding chromosome designation associated with the input feature representation segment; and
processing each segment-wise transformer input data object using the transformer-based machine learning model to generate the multi-segment input feature representation.

3. The computer-implemented method of claim 1, wherein the c input feature representation are generated based at least in part on a segmentation policy that defines: (i) for each chromosome designation, an intra-chromosome segment count, and (ii) a shared per-segment input feature representation value count that is common across the m input feature representation segments.

4. The computer-implemented method of claim 1, wherein each feature value is associated with an input feature type designation of a plurality of input feature type designations, and generating the initial input feature representation comprises:
generating one or more image representations of the input feature, wherein: (i) an image representation count of the one or more image representations is based at least in part on the plurality of input feature type designations, (ii) each image representation of the one or more image representations comprises a plurality of image regions, (iii) each image region for an image representation corresponds to a genetic variant identifier, and (iv) generating each of the one or more image representations associated with a character category is performed based at least in part on the one or more feature values of the input feature having the input feature type designation;
generating a tensor representation of the one or more image representations of the input feature;
generating, using the one or more processors, a plurality of positional encoding maps, wherein: (i) each positional encoding map of the one or more positional encoding maps comprises a plurality of positional encoding map regions, (ii) each positional encoding map region for a positional encoding map corresponds to a genetic variant identifier, (iii) each genetic variant identifier is associated with a positional encoding map region set comprising each positional encoding map region associated with the genetic variant identifier across the plurality of positional encoding maps, and (iv) each positional encoding map region set for a genetic variant identifier represents the genetic variant identifier;
generating the initial input feature representation based at least in part on the tensor representation and the plurality of positional encoding maps.

5. The computer-implemented method of claim 4, wherein generating the one or more image representations of the input feature further comprises:
generating a first image representation generated based at least in part on a first subset of input features;
generating a second image representation generated based at least in part on a second subset of input feature; and
generating a differential image representation of the one or more image representations based at least in part on performing an image difference operation across the first image representation and the second image representation.

6. The computer-implemented method of claim 4, wherein generating the one or more image representations of the input feature further comprises:
generating a first allele image representation generated based at least in part on a subset of the input features corresponding to a first allele;
generating a second allele image representation generated based at least in part on a subset of the input feature corresponding to a second allele;
generating a dominant allele image representation generated based at least in part on a subset of the input feature corresponding to a dominant allele;
generating a minor allele image representation generated based at least in part on a subset of the input feature corresponding to a minor allele; and
generating a zygosity image representation of the one or more image representations based at least in part on performing one or more operations across the first allele image representation, the second allele image representation, the dominant allele image representation, and the minor allele image representation.

7. The computer-implemented method of claim 4, wherein generating the one or more image representations of the input feature further comprises:
identifying one or more initial image representations of the input feature;
assigning one or more intensity values to each input feature type designation of the plurality of input feature type designations;
generating one or more intensity image representations of the one or more initial image representations, wherein (i) each image representation of the one or more intensity image representations comprises a plurality of intensity image regions, (ii) each image region for an intensity image representation corresponds to a genetic variant identifier, and (iii) generating the one or more intensity image representations is determined based at least in part on the one or more feature values and the assigned intensity value for each input feature type designation.

8. The computer-implemented method of claim 4, wherein the image-based prediction comprises generating, using the one or more processors, a polygenic risk score for one or more diseases for one or more individuals associated with the input feature.

9. The computer-implemented method of claim 4, wherein each feature value of the one or more feature values corresponds to a categorical feature type or numerical feature type.

10. An apparatus for generating a multi-segment prediction based at least in part on an initial input feature representation, the apparatus comprising at least one processor and at least one memory including program code, the at least one memory and the program code configured to, with the at least one processor, cause the apparatus to at least:
determine, based at least in part on the initial input feature representation, an ordered sequence of n input feature representation values, wherein: (i) the initial input feature representation is a fixed-size representation of an input feature comprising g feature values, (ii) each feature value corresponds to a genetic variant identifier of g genetic variant identifiers, (iii) each genetic variant identifier is associated with a chromosome designation of c chromosome designations and a corresponding variant-related subsequence of the ordered sequence, and (iv) each chromosome designation is associated with a chromosome-related subsequence of the ordered sequence;
generate, based at least in part on the ordered sequence, c input feature representation super-segments, wherein each input feature representation segment is associated with a corresponding chromosome designation and comprises the chromosome-related subsequence for the corresponding chromosome designation;
generate, based at least in part on the c input feature representation super-segments, m input feature representation segments of the ordered sequence, wherein the m input feature representation segments comprise, for each chromosome designation, a chromosome-related segment subset of the m input feature representation segments that comprises those input feature representation segments that are generated by segmentizing the input feature representation super-segment for the chromosome designation;
for each input feature representation segment, determine, using a shared segment embedding machine learning model and based at least in part on the input feature representation segment, a segment-wise representation of the input feature representation segment;
determine, using a transformer-based machine learning model and based at least in part on each segment-wise representation, a multi-segment input feature representation of the input feature;
generate, using the one or more processors and based at least in part on the multi-segment input feature representation and using a downstream prediction machine learning model, the multi-segment prediction; and
perform, using the one or more processors, one or more prediction-based actions based at least in part on the multi-segment prediction.

11. The apparatus of claim 10, wherein determining the multi-segment input feature representation comprises:
determining an ordered segment sequence of the m input feature representation segments based at least in part on the ordered sequence;
for each input feature representation segment, determining a segment-wise transformer input data object based at least in part on the segment-wise representation of the input feature representation segment, a positional representation of a segment in-sequence positional indicator for the input feature representation segment within the ordered segment sequence, and a chromosome representation of the corresponding chromosome designation associated with the input feature representation segment; and
processing each segment-wise transformer input data object using the transformer-based machine learning model to generate the multi-segment input feature representation.

12. The apparatus of claim 10, wherein the c input feature representation are generated based at least in part on a segmentation policy that defines: (i) for each chromosome designation, an intra-chromosome segment count, and (ii) a shared per-segment input feature representation value count that is common across the m input feature representation segments.

13. The apparatus of claim 10, wherein each feature value is associated with an input feature type designation of a plurality of input feature type designations, and generating the initial input feature representation comprises:
generating one or more image representations of the input feature, wherein: (i) an image representation count of the one or more image representations is based at least in part on the plurality of input feature type designations (ii) each image representation of the one or more image representations comprises a plurality of image regions, (iii) each image region for an image representation corresponds to a genetic variant identifier, and (iv) generating each of the one or more image representations associated with a character category is performed based at least in part on the one or more feature values of the input feature having the input feature type designation;
generating a tensor representation of the one or more image representations of the input feature;
generating, using the one or more processors, a plurality of positional encoding maps, wherein: (i) each positional encoding map of the one or more positional encoding maps comprises a plurality of positional encoding map regions, (ii) each positional encoding map region for a positional encoding map corresponds to a genetic variant identifier, (iii) each genetic variant identifier is associated with a positional encoding map region set comprising each positional encoding map region associated with the genetic variant identifier across the plurality of positional encoding maps, and (iv) each positional encoding map region set for a genetic variant identifier represents a the genetic variant identifier;
generating the initial input feature representation based at least in part on the tensor representation and the plurality of positional encoding maps.

14. The apparatus of claim 13, wherein generating the one or more image representations of the input feature further comprises:
generating a first image representation generated based at least in part on a first subset of input features;
generating a second image representation generated based at least in part on a second subset of input feature; and
generating a differential image representation of the one or more image representations based at least in part on performing an image difference operation across the first image representation and the second image representation.

15. The apparatus of claim 13, wherein generating the one or more image representations of the input feature further comprises:
generating a first allele image representation generated based at least in part on a subset of the input features corresponding to a first allele;
generating a second allele image representation generated based at least in part on a subset of the input feature corresponding to a second allele;
generating a dominant allele image representation generated based at least in part on a subset of the input feature corresponding to a dominant allele;
generating a minor allele image representation generated based at least in part on a subset of the input feature corresponding to a minor allele; and
generating a zygosity image representation of the one or more image representations based at least in part on performing one or more operations across the first allele image representation, the second allele image representation, the dominant allele image representation, and the minor allele image representation.

16. The apparatus of claim 13, wherein generating the one or more image representations of the input feature further comprises:
identifying one or more initial image representations of the input feature;
assigning one or more intensity values to each input feature type designation of the plurality of input feature type designations;
generating one or more intensity image representations of the one or more initial image representations, wherein (i) each image representation of the one or more intensity image representations comprises a plurality of intensity image regions, (ii) each image region for an intensity image representation corresponds to a genetic variant identifier, and (iii) generating the one or more intensity image representations is determined based at least in part on the one or more feature values and the assigned intensity value for each input feature type designation.

17. The apparatus of claim 13, wherein the image-based prediction comprises generating, using the one or more processors, a polygenic risk score for one or more diseases for one or more individuals associated with the input feature.

18. The apparatus of claim 13, wherein each feature value of the one or more feature values corresponds to a categorical feature type or numerical feature type.

19. A computer program product for generating a multi-segment prediction based at least in part on an initial input feature representation, the computer program product comprising at least one non-transitory computer-readable storage medium having computer-readable program code portions stored therein, the computer-readable program code portions configured to:
determine, based at least in part on the initial input feature representation, an ordered sequence of n input feature representation values, wherein: (i) the initial input feature representation is a fixed-size representation of an input feature comprising g feature values, (ii) each feature value corresponds to a genetic variant identifier of g genetic variant identifiers, (iii) each genetic variant identifier is associated with a chromosome designation of c chromosome designations and a corresponding variant-related subsequence of the ordered sequence, and (iv) each chromosome designation is associated with a chromosome-related subsequence of the ordered sequence;
generate, based at least in part on the ordered sequence, c input feature representation super-segments, wherein each input feature representation segment is associated with a corresponding chromosome designation and comprises the chromosome-related subsequence for the corresponding chromosome designation;
generate, based at least in part on the c input feature representation super-segments, m input feature representation segments of the ordered sequence, wherein the m input feature representation segments comprise, for each chromosome designation, a chromosome-related segment subset of the m input feature representation segments that comprises those input feature representation segments that are generated by segmentizing the input feature representation super-segment for the chromosome designation;
for each input feature representation segment, determine, using a shared segment embedding machine learning model and based at least in part on the input feature representation segment, a segment-wise representation of the input feature representation segment;
determine, using a transformer-based machine learning model and based at least in part on each segment-wise representation, a multi-segment input feature representation of the input feature;
generate, using the one or more processors and based at least in part on the multi-segment input feature representation and using a downstream prediction machine learning model, the multi-segment prediction; and
perform, using the one or more processors, one or more prediction-based actions based at least in part on the multi-segment prediction.

20. The computer program product of claim 19, wherein determining the multi-segment input feature representation comprises:
determining an ordered segment sequence of the m input feature representation segments based at least in part on the ordered sequence;
for each input feature representation segment, determining a segment-wise transformer input data object based at least in part on the segment-wise representation of the input feature representation segment, a positional representation of a segment in-sequence positional indicator for the input feature representation segment within the ordered segment sequence, and a chromosome representation of the corresponding chromosome designation associated with the input feature representation segment; and
processing each segment-wise transformer input data object using the transformer-based machine learning model to generate the multi-segment input feature representation.
